(19) United States
(12) Patent Application Publication
(10) Pub. No.: US 2023/0041692 A1
(43) Pub. Date: Feb. 9, 2023

(54) NOVEL PEPTIDE HORMONES

(71) Applicant: THE UNIVERSITY OF SYDNEY, Sydney, NSW (AU)

(72) Inventor: Mark LARANCE, Sydney, NSW (AU)

(21) Appl. No.: 17/757,016

(22) PCT Filed: Dec. 9, 2020
(86) PCT No.: PCT/AU2020/051345

§ 371 Date: Jun. 8, 2022

(30) Foreign Application Priority Data
Dec. 9, 2019 (AU) 2019904654

(51) Int. Cl. C07K 7/08; C07K 16/26; C07K 14/575; C12N 15/63

(52) U.S. Cl. CPC C07K 7/08; C07K 14/575; C07K 16/26; C12N 15/63

(57) ABSTRACT

The present invention relates to peptides that are upregulated in response to nutritional cues, antibodies which bind to the peptides, nucleic acids encoding the peptides, vectors and host cells.

### INCORPORATION BY CROSS-REFERENCE

This application claims priority from Australian provisional patent application number 2019904654, filed on 9 Dec. 2019, the entire contents of which are incorporated herein by cross-reference.

### FIELD OF THE INVENTION

The present invention relates generally to the fields of cell biology, molecular biology and endocrinology. More specifically, the present invention relates to peptides that are upregulated in response to nutritional cues and are postulated to have hormonal activity.

### BACKGROUND TO THE INVENTION

The following discussion of the background of the invention is merely provided to aid the reader in understanding the invention, and is not admitted to describe or constitute prior art to the invention.

Hormones are chemical messengers produced by the body which help maintain homeostasis and play an important role in the regulation of processes such as reproduction and metabolism as well as states such as mood and appetite. Hormone discoveries have led to enormous benefits to human health. The discovery of insulin about one hundred years ago has been amongst the greatest biological discoveries to date and has subsequently saved millions of lives. More recent discoveries of gut-derived hormones such as GLP-1 (glucagon-like peptide 1), which is secreted into the blood after feeding, have also had a significant therapeutic impact in diseases such as type II diabetes. Administration of GLP-1 or its analogues can supress appetite through signalling to the hypothalamus, alongside many other clinically beneficial effects on other organ systems. The GLP-1 peptide and other similar hormones are secreted from cells in the intestine called enteroendrocrine cells. These are rare cells (approximately 1 in every 100 cells) that respond to food intake to either stop, or to start, hormone secretion into the blood. Ghrelin is one of the few peptide hormones secreted by the gut (stomach) to increase appetite when no food has been consumed (fasting). Due to its ability to stimulate appetite ghrelin has been trialled for the treatment of cachexia (body wasting) related to cancer and other diseases.

Insulin, GLP-1 and ghrelin are examples of small-protein hormones, also commonly known as peptide hormones. The discovery of peptide hormones enables them to be isolated from their natural sources and purified for use as either replacements, or supplements in cases where endogenous levels are absent or insufficient.

Advances in technology since hormones were first discovered have enabled the sequencing and chemical synthesis of peptide hormones, leading to the development of drugs which are analogs of natural peptides with improved pharmaceutical properties and/or peptides conjugated to molecules useful for, for example, tissue-specific targeting or extension of half-life. Advances in genomics have led to the elucidation of pathways involving hormones and the identification of cognate receptors which can be manipulated with either natural or synthetic agonists or antagonists to restore, enhance or remove function. Global changes in peptide hormone levels during many key immunological, metabolic and developmental perturbations make them useful markers for diagnosis and/or prognosis.

Obesity, defined by the World Health Organization as an abnormal or excessive fat accumulation that presents a risk to health, is a risk factor for many chronic diseases such as diabetes, cardiovascular disease and cancer. Rates of obesity are increasing worldwide, prompting an increased interest in the processes controlling appetite regulation. The discovery of GLP-1 led to the development of therapeutics for obesity, type 2 diabetes, high blood pressure and high cholesterol. However, since the discovery of GLP-1 and related hormones decades ago, the discovery of new hormones in more recent times has been slow.

Cachexia is a common factor that occurs during the treatment for many cancers, but can also occur due to psychological disorders such as anorexia nervosa. In both of these diseases the cachexia can be severe and may lead to death. Therefore, many treatments have been sought to increase the appetite of these patients.

There is an unmet need for the discovery and characterisation of new peptide hormones involved in homeostasis, appetite regulation, the maintenance of fat mass, glycaemic control and the responses to feeding and fasting.

### SUMMARY OF THE INVENTION

The present invention meets at least one of the needs mentioned above by providing novel, previously uncharacterised peptides, which may be upregulated in response to a mixed food meal and are postulated to have hormonal activity. In Australian Provisional Patent Application No. 2019901536, the entire disclosure of which is incorporated herein by cross-reference, the present inventor discloses methods for the fractionation of low molecular weight proteins from mixed protein populations. Using these methods, the present inventor has identified novel peptides. The novel peptides may exist as several isoforms. The peptides of the present invention, and isoforms of said peptides, may be referred to herein as “erusiolin”.

Experimental data provided herein demonstrates that the peptides of the present inventio n are significantly increased in abundance following a period of intermittent fasting. Accordingly, the inventor has hypothesised that the peptides may have hormonal activity and could potentially be manipulated to stimulate or suppress appetite. Without limitation, the peptides described herein may function as hormones and may generally be useful in the regulation of appetite in subjects in need of weight management.

The present invention relates at least in part to the following embodiments:

In a first aspect, the present invention provides a peptide comprising or consisting of the amino acid sequence
X₁ X₂ X₃ X₄ X₅ X₆ X₇ X₈ X₉ X₁₀ X₁₁ X₁₂ X₁₃ X₁₄ X₁₅ X₁₆ X₁₇
wherein X₁, X₂, X₁₆ and X₁₇ are optionally present, and wherein:
X₁ is selected from A, L, V, and I, or is absent;
X₂ is selected from P and S, or is absent;
X₃ is selected from F and V;
X₄ is selected from L, M, V, I, A and F;
X₅ is selected from L, M, S, K, L, A and T;
X₆ is selected from E, Q, G and P;
X₇ is selected from D, Q, K and E;
X₈ is selected from P, S, E, A, Q and T;
X₉ is selected from A, S, D, T and V;
X₁₀ is selected from N and K;
X₁₁ is selected from Q, R, G, K and E;
X₁₂ is selected from F and I;
X₁₃ is selected from L, I and M;
X₁₄ is selected from R, H, G and Q;
X₁₅ is selected from L, M, H, F and Q;
X₁₆ is K or is absent; and
X₁₇ is R or is absent.

In one embodiment of the first aspect,
X₁ is A, or is absent;
X₂ is P, or is absent;
X₃ is F;
X₆ is E;
X₉ is A;
X₁₂ is F;
X₁₄ is R; and
X₁₅ is L.

In one embodiment of the first aspect,
X₄ is L;
X₁₀ is N;
X₁₁ is Q; and
X₁₃ is L.

In one embodiment of the first aspect, the peptide comprises or consists of an amino acid sequence as defined in any one of SEQ ID NO: 1, SEQ ID NO: 2, SEQ ID NO: 3 or SEQ ID NO: 4.

In one embodiment of the first aspect, the peptide comprises one or more modifications to the chemical structure of one or more amino acids and/or peptide bonds.

In one embodiment of the first aspect, the modification/s comprise:
N-terminal acetylation; and/or
N-methylation of peptide bonds and/or
replacement of an L-stereoisomer amino acid residue with the equivalent D-stereoisomer.

In one embodiment of the first aspect, the modification/s protect a peptide bond from cleavage by a protease.

In one embodiment of the first aspect, the peptide is a synthetic peptide.

In one embodiment of the first aspect, the peptide is a hormone and/or is capable of hormone activity.

In one embodiment of the first aspect, the hormone is capable of appetite regulation.

In one embodiment of the first aspect, the hormone produces satiety (fullness) signals.

In one embodiment of the first aspect, the peptide has at least 70%, at least 80%, at least 85%, at least 90%, at least 95% or 100% of the biological activity of a peptide having an amino acid sequence as defined in any one of SEQ ID NO: 1, SEQ ID NO: 2, SEQ ID NO: 3 or SEQ ID NO: 4.

In one embodiment of the first aspect, the peptide has an increased biological activity of at least 10%, at least 20%, at least 30%, at least 40%, at least 50%, at least 60%, at least 70%, at least 80%, at least 90%, or at least 100% above the biological activity of a peptide having an amino acid sequence as defined in any one of SEQ ID NO: 1, SEQ ID NO: 2, SEQ ID NO: 3 or SEQ ID NO: 4.

In a second aspect, the present invention provides an antibody that binds specifically to the peptide of the first aspect.

In one embodiment of the second aspect, the antibody is a polyclonal antibody.

In one embodiment of the second aspect, the polyclonal antibody is a rabbit polyclonal antibody.

In one embodiment of the second aspect, the antibody is a monoclonal antibody.

In one embodiment of the second aspect, the monoclonal antibody is a rabbit monoclonal antibody.

In a third aspect, the present invention provides a nucleic acid molecule encoding the peptide according to the first aspect, wherein the nucleic acid molecule is operably linked to at least one heterologous regulatory element.

In one embodiment of the third aspect, the nucleic acid molecule comprises at least one modification to enhance expression of the nucleic acid molecule in a heterologous host.

In one embodiment of the third aspect, the at least one modification optimises the use of codons to enhance expression of the nucleic acid molecule in the heterologous host.

In a fourth aspect, the present invention provides a vector comprising the nucleic acid molecule according to the third aspect.

In a fifth aspect, the present invention provides a host cell comprising the nucleic acid molecule according to the third aspect as a transgene and/or the vector according to the fourth aspect.

### DEFINITIONS

Certain terms are used herein which shall have the meanings set forth as follows.

As used in this application, the singular form “a”, “an” and “the” include plural references unless the context clearly dictates otherwise. For example, the term “peptide” also includes a plurality of peptides unless otherwise stated.

As used herein, the term “comprising” means “including”. Variations of the word “comprising”, such as “comprise” and “comprises” have correspondingly varied meanings. Thus, for example, a sequence “comprising” may consist exclusively of or may include one or more additional amino acids or nucleotides.

As used herein, the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any numerical value derivable therein, and any range derivable therein.

As used herein, the term “between” when used in reference to a range of numerical values encompasses the numerical values at each endpoint of the range.

As used herein, the terms “protein” and “peptide” each refer to a polymer made up of amino acids linked together by peptide bonds and are used interchangeably. For the purposes of the present invention a “peptide” may constitute a full-length protein or a portion of a full-length protein.

As used herein, the term “synthetic”, when used to describe a product, refers to a product produced by human agency as opposed to a naturally occurring product. For example, a “synthetic” peptide refers to a peptide which has been produced by artificial chemical reactions.

As used herein a “conservative” amino acid substitution refers to a scenario where an amino acid residue in a base peptide sequence is substituted by another amino acid residue having a side chain (R group) with similar biochemical properties (e.g. charge and/or hydrophobicity and/or size) that does not substantially change the biological activity compared to the base peptide.

As used herein a “non-conservative” amino acid substitution refers to a scenario where an amino acid residue in a base peptide sequence is substituted by another amino acid residue having a side chain (R group) with different biochemical properties (e.g. charge and/or hydrophobicity and/or size) which result in at least some alteration in the biological activity compared to the base peptide.

As used herein a molecule that “binds specifically to” another molecule is one with binding specificity for that different molecule. For example, if molecule A “binds specifically to” molecule B, molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by the reagent. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level For example, an an antibody that binds specifically to a peptide a having the amino acid sequence of SEQ ID NO: 1 will preferentially bind to a peptide having the amino acid sequence of SEQ ID NO: 1 at least 10-fold, preferably 50-fold, more preferably 100-fold, and most preferably greater than 100-fold more frequently than other potential binding partners.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### ABBREVIATIONS

The following abbreviations are used herein and throughout the specification:
µg: microgram/s
µL: microlitre/s
µm: micrometre/s
Å: angstrom
ACN: acetonitrile
BLAST: Basic Local Alignment Search Tool
BMI: body mass index
C terminal: carboxy terminal
C-terminus: carboxyl-terminus
CPE: carboxypeptidase E
C3ORF85: chromosome 3 open reading frame 85
DIA: data-independent acquisition
DPP4: dipeptidyl peptidase 4
DTT: dithiothreitol
EDTA: ethylenediaminetetraacetic acid
ELISA: enzyme- linked immunosorbent assay
FDR: false discovery rate
g: gravitational force
GIP: gastric inhibitory polypeptide
GLP-1: glucagon-like peptide 1
h: hour/s
H₂SO₄: sulfuric acid
H₃PO₄: phosphoric acid
HCl: hydrochloric acid
HMDP: hybrid mouse diversity panel
HNO₃: nitric acid
HPLC: high pressure liquid chromatography
IF: intermittent fasting
Ig: immunoglobulin
IGF-1: insulin- like growth factor 1
IL-36gamma: interleukin 36 gamma
iRT: indexed retention time
kDa: kilodalton/s
KLH: keyhole limpet hemocyanin
LC: liquid chromatography
LC-MS/MS: liquid chromatography-tandem mass spectrometry
M: molar
min: minute/s
mL: millilitre/s
mm: millimetre/s
MRM: multiple reaction monitoring
MSIA: mass spectrometry immunoassay
m/z: mass-to-charge ratio
ng: nanogram/s
nm: nanometre/s
NMR: nuclear magnetic resonance
NPY: neuropeptide Y
N terminal: amino terminal
MS/MS: tandem mass spectrometry
PCR: polymerase chain reaction
PRM: parallel reaction monitoring
PWS: Prader-Willi syndrome
RANTES: Regulated upon Activation, Normal T cell Expressed, and Secreted
rpm: revolutions per minute
s: seconds
SDF-1: stromal cell-derived factor 1
SEC: size exclusion chromatography
SRM: selected reaction monitoring
TEAB: triethylammonium bicarbonate
TCEP: tris-(2-carboxyethyl) phosphine
TFA: trifluoroacetic acid
Th: theoretical fragment ion mass spectra
THPP: tris-(3-hydroxypropyl) phosphine
UHPLC: ultra high pressure liquid chromatography
UV: ultraviolet

### BRIEF DESCRIPTION OF THE FIGURES

Preferred embodiments of the present invention will now be described, by way of example only, with reference to the accompanying Figures as set out below:

FIG. 1: Erusiolin Protein Structure and Quantification. FIG. 1a provides the protein structure and protease cleavage sites predicted from sequence analysis and alignment across species. See FIG. 2 for full protein sequence alignment across species. FIG. 1b shows a comparison of the abundance of erusiolin-derived peptides in fasted human blood plasma from 22 patients before and after 8 weeks of IF. Each patient is indicated by a dot. The control peptide is from the non-conserved region of C3ORF85.

FIG. 2: C3ORF85 Homologue Protein Sequence Alignment. FIG. 2 provides a multiple sequence alignment of protein sequences encoded by homologues of C3ORF85.

FIG. 3: C3ORF85 Conserved Peptide Alignment. FIG. 3a provides a multiple sequence alignment of the conserved region of the protein sequences encoded by homologues of C3ORF85.FIG. 3b provides a prediction of the effect of mutating each wild type residue to each of the 20 amino acids. FIG. 3c provides a sequence logo for the full-length erusiolin peptide where the height of each amino acid symbol letter correlates with the conservation of that amino acid in that position across evolution, taller letters being more conserved.

FIG. 4: Erusiolin (human C3ORF85 conserved sequence) antibody generation and its application to identify major circulating forms of the hormone in human blood plasma. FIG. 4a shows the peptide sequence used to generate rabbit polyclonal antibodies. FIG. 4b shows the results from testing the antibody performance and titre using an enzyme-linked immunosorbent assay (ELISA). In FIG. 4c, the antibody was employed in a mass spectrometry immunoassay (MSIA) on human plasma to isolate all of the circulating forms of the peptide, which were subsequently identified by liquid chromatography (LC) coupled to tandem mass spectrometry (MS/MS). FIG. 4d provides a plot of the intensity (relative abundance) for some of the most abundant forms of the erusiolin conserved peptide sequence identified by mass spectrometry in human plasma. FIG. 4e provides the annotated MS/MS spectra for the most abundant form of the hormone identified in human plasma. Peptide fragment y ions and b ions are shown.

FIG. 5: mRNA Abundance of erusiolin in human and mouse tissues using publicly available datasets. FIG. 5a erusiolin (C3ORF85) mRNA abundance across human tissues (Human Protein Atlas, https://www.proteinatlas.org/). FIG. 5b erusiolin (Gm5485) mRNA abundance across mouse tissues with data from 3 animals per tissue shown (NCBI GEO Dataset: GDS3142).

FIG. 6: Analysis of erusiolin producing cells, secretion in response to food and effects of erusiolin injection in mice. FIG. 6a shows fluorescent staining of human duodenal tissue for the erusiolin peptide using the rabbit polyclonal antibody and for genomic DNA A rectangle highlights the erusiolin peptide in the top panel. FIG. 6b shows fluorescent co-staining of human duodenal tissue for the erusiolin peptide using the rabbit polyclonal antibody, the enteroendocrine cell marker gastric inhibitory polypeptide (GIP), and for genomic DNA. Staining is consistent with erusiolin positivity in rare enteroendocrine cell populations. FIG. 6c shows the abundance measurement of peptide hormones in human plasma before and after a mixed meal test. Each line is one participant. FIG. 6d shows a crossover trial of intraperitoneal peptide injection in C57BL/6J male mice at 14 weeks of age. Animals were randomised to the sequence of treatment days that were random in order, with washout days between injection treatment days. Peptides were injected at 5pm before the night phase and the change in body weight and food intake measured after 16 h. (n=14) ***P<0.001,*P<0.05. Erusiolin refers to the synthetic peptide of sequence APFLLEDPANQFLRL (SEQ ID NO: 2), DPP4-cleaved refers to the synthetic peptide of sequence FLLEDPANQFLRL (SEQ ID NO: 3).

FIG. 7: Erusiolin is linked to hypothalamus specific Prader-Willi syndrome (PWS) locus transcription. FIG. 7a shows Quantitative Endocrine Network Interaction Estimation for the mouse erusiolin gene (Gm5485). Plotted here are hypothalamus-derived transcripts that were linked with erusiolin and have a Bonferroni-corrected P< 1e-4. The odds ratio for linkage of each transcript to one of three intronic SNPs in the erusiolin locus is shown on the x-axis. Black circles are part of the PWS locus. FIG. 7b provides a schematic of a hypothesised function of erusiolin to inhibit food intake.

FIG. 8: Human erusiolin (full-length) structural analysis. FIG. 8a Far-UV circular dichroism (CD) spectra of human C3ORF85 sequence between the signal peptide cleavage site and the furin cleavage site (APFLLEDPANQFLRLKR). Proportion of helical structure is indicated by the minima at 208 and 222 nm. FIG. 8b provides a predicted structure of the human C3ORF85 sequence between the signal peptide cleavage site and the furin cleavage site (APFLLEDPANQFLRLKR), analysed using PEP-FOLD3. This analysis indicated a C-terminal alpha-helix within the sequence ANQFLRLKR. FIG. 8c ¹H-NMR chemical shift and NOE analysis confirms the presence of a C-terminal alpha-helix. The chemical shifts of alpha carbon protons are known to be predictive of secondary structure and comparison of the observed shifts with those of an unstructured ‘standard’ peptide enable detection of a tendency for helical structure (CSI, H = helix). NOEs refer to interactions of the protons through space and are typically limited to maximum distances of 5 angstroms. ‘NN’ refers to the amide proton of the i residue to the amide proton of the (i+n) residue, where n is a positive integer; ‘alphaN’ refers to the alpha carbon proton of i to the amide proton of (i+n); ‘betaN’ refers to the beta carbon proton(s) of i to the amide proton of (i+n).

### DETAILED DESCRIPTION OF THE INVENTION

### Erusiolin

The present invention provides novel, previously uncharacterised peptides. In some embodiments, the peptides of the present invention exist as isoforms. The peptides of the present invention and all isoforms of the peptides may be referred to herein as “erusiolin”. In some embodiments, the novel peptides may function as peptide hormones. In a further embodiment, the present invention provides a peptide which comprises or consists of the amino acid sequence APFLLEDPANQFLRLKR (SEQ ID NO: 1), APFLLEDPANQFLRL (SEQ ID NO: 2), FLLEDPANQFLRL (SEQ ID NO: 3) or FLLEDPANQFLRLKR (SEQ ID NO: 4). In still other embodiments, a peptide of the present invention may comprise or consist of an amino acid sequence having a sequence identity of at least 70%, at least 80%, at least 90%, at least 95%, at least 99% or 100% to SEQ ID NO. 1, SEQ ID NO: 2, SEQ ID NO: 3, and/or SEQ ID NO: 4.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences being compared. For example, an amino acid sequence (“subject sequence”) having at least 95% “sequence identity” with another amino acid sequence (“query sequence”) is intended to mean that the subject sequence is identical to the query sequence except that the subject sequence may include up to five amino acid alterations per 100 nucleotides of the query sequence. In other words, to obtain an amino acid sequence of at least 95% sequence identity to a query sequence, up to 5% (i.e. 5 in 100) of the amino acids in the subject sequence may be inserted, substituted with another amino acid or deleted.

Methods for assessing the level of homology and identity between sequences are well known in the art. The percentage of sequence identity between two sequences may, for example, be calculated using a mathematical algorithm. A non-limiting example of a suitable mathematical algorithm is described in the publication of Karlin and colleagues (1993, PNAS USA, 90:5873-5877). This algorithm is integrated in the BLAST (Basic Local Alignment Search Tool) family of programs (see also Altschul et al. 1990 J. Mol. Biol. 215, 403-410 or Altschul et al. 1997 Nucleic Acids Res, 25:3389-3402) accessible via the National Center for Biotechnology Information (NCBI) website homepage (https://www.ncbi.nlmnih.gov). The BLAST program is freely accessible at https://blast.ncbi.nlm.nih.gov/Blast.cgi. Other non-limiting examples include the Clustal (http://www.clustalorg/) and FASTA (Pearson 1990 Methods Enzymol. 83, 63-98; Pearson and Lipman 1988 Proc. Natl. Acad. Sci. U. S. A 85, 2444-2448.) programs. These and other programs can be used to identify sequences which are at least to some level identical to a given input sequence. Additionally or alternatively, programs available in the Wisconsin Sequence Analysis Package, version 9.1 (Devereux et al. 1984 Nucleic Acids Res., 387-395), for example the programs GAP and BESTFIT, may be used to determine the percentage of sequence identity between two polypeptide sequences. BESTFIT uses the local homology algorithm of Smith and Waterman (1981, J. Mol. Biol. 147, 195- 197) and identifies the best single region of similarity between two sequences. Where reference herein is made to an amino acid sequence sharing a specified percentage of sequence identity to a reference amino acid sequence, the difference/s between the sequences may arise partially or completely from conservative amino acid substitution/s. In such cases, the sequence identified with the conservative amino acid substitution/s may substantially or completely retain the same biological activity of the reference sequence.

Peptides according to the present invention may comprise of consist of 1, 2, 3 or more amino acid substitution/s when compared to the amino acid sequence as defined in any one or more of SEQ ID NO. 1, SEQ ID NO: 2, SEQ ID NO: 3, and/or SEQ ID NO:4.

Any one or more of the 1, 2, 3 or more amino acid substitutions may be conservative amino acid substitution/s in which a given amino acid residue is replaced with an amino acid residue having a side chain with similar biochemical properties including, but not limited to, charge, hydrophobicity and/or size. Families of amino acid residues having similar side chains are well known in the art. By way of non-limiting example, conservative amino acid substitution/s may involve substituting amino acids within the following groupings: aliphatic (ILV); hydrophobic (F W Y H K M I L V A G C); polar (W Y H K R E D C S T N Q); small (V C A G S P T N D); tiny (A G S); charged (H K R E D); negatively charged (E D); positively charged (H K R); aromatic (F W Y H). By way of further non-limiting example, the conservative amino acid substitution/s may be made at any of residues 1-17 of SEQ ID NO: 1, residues 1-15 of SEQ ID NO: 2, residues 1-13 of SEQ ID NO: 3, or residues 1-15 of SEQ ID NO: 4. Peptides with 1, 2, 3 or more conservative amino acid substitution/s may maintain the same or similar biological activity of the base peptide (i.e. the biological activity of a peptide comprising or consisting of an amino acid sequence as defined in SEQ ID NO. 1, SEQ ID NO: 2, SEQ ID NO: 3, and/or SEQ ID NO:4).

Additionally, or alternatively, any one or more of the 1, 2, 3 or more amino acid substitutions may be a non-conservative amino acid substitution in which a given amino acid residue is replaced with an amino acid residue having a side chain with different biochemical properties including, but not limited to charge, hydrophobicity and/or size. By way of non-limiting example, non-conservative amino acid substitution/s may involve substituting amino acids between any of the following groupings: aliphatic (I L V); hydrophobic (F W Y H KM I L V A G C); polar (W Y H K R E D C S T N Q); small (V C A G S P T N D); tiny (A G S); charged (H K R E D); negatively charged (E D); positively charged (H K R); aromatic (F W Y H). By way of further non-limiting example, the non-conservative amino acid substitution/s may be made at any of residues 1-17 of SEQ ID NO: 1, residues 1-15 of SEQ ID NO: 2, residues 1-13 of SEQ ID NO: 3, or residues 1-15 of SEQ ID NO: 4. Peptides with 1, 2, 3 or more non-conservative amino acid substitution/s may have at least some alteration (e.g. enhancement or reduction) in biological activity compared to the base peptide (i.e. the biological activity of a peptide comprising or consisting of an amino acid sequence as defined in SEQ ID NO. 1, SEQ ID NO: 2, SEQ ID NO: 3, and/or SEQ ID NO:4).

Peptides of the present invention may maintain at least 70%, at least 80%, at least 85%, at least 90%, at least 95% or 100% of the biological activity of the base peptide following amino acid substitution/s. Additionally or alternatively, the biological activity of peptides may increase by at least 10%, at least 20%, at least 30%, at least 40%, at least 50%, at least 60%, at least 70%, at least 80%, at least 90%, or at least 100% of the activity of the base peptide following the amino acid substitution/s. In some embodiments of the present invention, the peptide may be described by the sequence X₁ X₂ X₃ X₄ X₅ X₆ X₇ X₈ X₉ X₁₀ X₁₁ X₁₂ X₁₃ X₁₄ X₁₅ X₁₆ X₁₇, wherein:
X₁ is selected from A, L, V, and I, or is absent;
X₂ is selected from P and S, or is absent;
X₃ is selected from F and V;
X₄ is selected from L, M, V, I, A and F;
X₅ is selected from L, M, S, K, L, A and T;
X₆ is selected from E, Q, G and P;
X₇ is selected from D, Q, K and E;
X₈ is selected from P, S, E, A, Q and T;
X₉ is selected from A, S, D, T and V;
X₁₀ is selected from N and K;
X₁₁ is selected from Q, R, G, K and E;
X₁₂ is selected from F and I;
X₁₃ is selected from L, I and M;
X₁₄ is selected from R, H, G and Q;
X₁₅ is selected from L, M, H, F and Q;
X₁₆ is K or is absent; and
X₁₇ is R or is absent.

In some embodiments, X₃ is F, X₆ is E, X₉ is A, X₁₂ is F, X₁₄ is R and/or X₁₅ is L.

The peptide may have the sequence:
A P F X₄ X₅ E X₇ X₈ A X₁₀ X₁₁ F X₁₃ R L K R, or
A P F X₄ X5 E X₇ X₈ A X₁₀ X₁₁ F X₁₃ R L, or
F X₄ X₅ E X₇ X₈ A X₁₀ X₁₁ F X₁₃ R L, or
F X₄ X₅ E X₇ X₈ A X₁₀ X₁₁ F X₁₃ R L K R, where:
X₄ is selected from L, M, V, I, A and F;
X₅ is selected from L, M, S, K, L, A and T;
X₇ is selected from D, Q, K and E;
X₈ is selected from P, S, E, A, Q and T;
X₁₀ is selected from N and K;
X₁₁ is selected from Q, R, G, K and E; and
X₁₃ is selected from L, I and M.

In other embodiments, the peptide may have the sequence:
A P F L X₅ E X₇ X₈ A N Q F X₁₃ R L K R, or
A P F L X₅ E X₇ X₈ A N Q F X₁₃ R L, or
F L X₅ E X₇ X₈ A N X₁₁ F Q R L, or
F L X₅ E X₇ X₈ A N X₁₁ F Q R L K R, where:
X₅ is selected from L, M, S, K, L, A and T;
X₇ is selected from D, Q, K and E;
X₈ is selected from P, S, E, A, Q and T.

### Identification of Erusiolin in a Biological Sample

In some embodiments of the present invention, erusiolin may be identified in a biological sample by methods disclosed by the present inventor in Australian Provisional Patent Application No. 2019901536, the entire disclosure of which is incorporated herein by cross-reference. Methods for the fractionation of low molecular weight proteins from mixed protein populations disclosed in Australian Provisional Patent Application No. 2019901536 may be used to separate peptide hormones such as erusiolin from larger proteins present in a biologic al sample.

### Protein-Protein Dissociation and Protein Precipitation

In some embodiments, protein-protein dissociation and protein precipitation is achieved by incubation of the biological sample in an extraction buffer to provide an extraction mixture. The extraction buffer may comprise HCl and ethanol In some embodiments, the concentration of HCl in the extraction buffer is between 0.05 M and 0.5 M, between 0.1 M and 0.4 M, between 0.2 M and 0.3 M, or between 0.2 M and 0.25 M. In some embodiments, the HCl may be replaced by H₂SO₄, HNO₃, H₃PO₄ or a mixture of any two or more of these acids. In some embodiments, the concentration of ethanol in the extraction buffer is under 95% (v/v), under 90% (v/v), under 80% (v/v), under 70% (v/v), under 60% (v/v), under 50% (v/v). In some embodiments, the ethanol may be replaced by methanol, or 1-propanol, or 2-propanol, or a mixture of any two or more of these alcohols. In one embodiment, the extraction buffer comprises 0.25 M HCl and 87.5% ethanol in H₂O. In some embodiments, the ratio of ethanol to 0.25 M HCl in the extraction buffer is 9:1, 8:1, 7:1, 6:1, 5:1 or 4:1. In one embodiment, the ratio of ethanol to 0.25 M HCl in the extraction buffer is 7:1.

In some embodiments, the volume of biological sample in the extraction mixture is under 40%, under 30%, under 20%, under 10%, or under 5% of the total volume of the incubation. In one embodiment, the biological sample comprises 10% of the total volume of the incubatio n.

In some embodiments, biological samples are obtained and immediately incubated in the extraction buffer. In some embodiments, biological samples are refrigerated prior to incubation in the extraction buffer. In some embodiments, biological samples are frozen, and must be thawed prior to incubation in the extraction buffer. A person skilled in the art would use a technique such as vortexing the biological sample as a matter of routine to ensure adequate mixing of the thawed biological sample prior to incubation.

In some embodiments, the biological sample is incubated in the extraction buffer at room temperature. In some embodiments, the biological sample is incubated in the extraction buffer for under 120 minutes, under 110 minutes, under 100 minutes, under 90 minutes, under 80 minutes, under 70 minutes, under 60 minutes, under 50 minutes, under 40 minutes, under 30 minutes, under 20 minutes, under 15 minutes, or under 10 minutes. In one embodiment, the biological sample is incubated in the extraction buffer for 30 minutes.

In some embodiments, precipitated proteins are pelleted by centrifugation. In one embodiment, precipitated proteins are pelleted by centrifugation at room temperature.

In further embodiments, either chloroform, or ethylacetate, or methyl tert-butyl ether, is added to the supernatant to ensure lipid removal prior to SEC-based separations.

### Size Exclusion Chromatography

Size exclusion chromatography has been used since the 1950s for numerous applications and with a wide range of analytes (Lindqvist and Storgards 1955 Nature 175: 511-512). A person skilled in the art would recognise that many variations of size exclusion chromatography exist which can be used to extract and separate proteins of interest. In some embodiments, size exclusion chromatography is denaturing size exclusion chromatography. In some embodiments, a UHPLC system is used in combination with size exclusion chromatography. In some embodiments, field-flow fractionation, or ultrafiltration, or capillary electrophoresis, or a combination of any two or more of these methods is used instead of size exclusion chromatography.

In some embodiments, size exclusion chromatography uses columns of pore size under 500 Å, under 300 Å, under 200 Å, or under 100 Å. In one embodiment, size exclusion chromatography uses a column of pore size 130 Å. In some embodiments, size exclusion chromatography uses columns of length under 300 mm, under 200 mm, or under 100 mm. In one embodiment, size exclusion chromatography uses a column of length 300 mm. In some embodiments, size exclusion chromatography uses columns of diameter under 30 mm, under 10 mm, or under 5 mm. In one embodiment, size exclusion chromatography uses a column of diameter 7.8 mm. In some embodiments, size exclusion chromatography uses columns with particles of diameter under 20 µm, under 10 µm, under 5 µm, or under 2 µm. In one embodiment, size exclusion chromatography uses a column with particles of diameter 2.7 µm.

In some embodiments, the SEC running buffer used comprises water, acetonitrile and TFA. In some embodiments, the concentration of acetonitrile in the SEC running buffer is under 50% (v/v), under 40% (v/v), under 30% (v/v), under 20% (v/v), or under 10% (v/v). In some embodiments, the acetonitrile may be replaced by methanol, ethanol, 1-propanol, or 2-propanol, or a mixture of any two or more of these solvents. In one embodiment, the concentration of acetonitrile in the SEC running buffer is 30% (v/v). In some embodiments, the concentration of TFA in the SEC running buffer is under 5% (v/v), under 1% (v/v), under 0.1% (v/v), or under 0.01% (v/v). In one embodiment, the concentration of TFA in the SEC running buffer is 0.1% (v/v). In some embodiments, the TFA may be replaced by formic acid, acetic acid, or heptafluorobutyric acid, or a mixture of any two or more of these acids.

In some embodiments, the fraction collected in size exclusion chromatography comprises or consists of proteins of molecular weight under 15 kDa, under 14 kDa, under 13 kDa, under 12 kDa, under 11 kDa, under 10 kDa, under 9 kDa, under 8 kDa, under 7 kDa, under 6 kDa, under 5 kDa, under 4 kDa, or under 3 kDa. In some embodiments, the fraction comprises or consists of proteins of molecular weight between 15 kDa and 2 kDa, between 14 kDa and 2 kDa, between 13 kDa and 2 kDa, between 12 kDa and 2 kDa, between 11 kDa and 2 kDa, between 10 kDa and 2 kDa, between 9 kDa and 2 kDa, between 8 kDa and 2 kDa, or between 7 kDa and 2 kDa. In some embodiments, the fraction comprises or consists of proteins of molecular weight between 15 kDa and 3 kDa, between 14 kDa and 3 kDa, between 13 kDa and 3 kDa, between 12 kDa and 3 kDa, between 11 kDa and 3 kDa, between 10 kDa and 3 kDa, between 9 kDa and 3 kDa, between 8 kDa and 3 kDa, or between 7 kDa and 3 kDa. In some embodiments, the fraction comprises or consists of proteins of molecular weight between 15 kDa and 4 kDa, between 14 kDa and 4 kDa, between 13 kDa and 4 kDa, between 12 kDa and 4 kDa, between 11 kDa and 4 kDa, between 10 kDa and 4 kDa, between 9 kDa and 4 kDa, between 8 kDa and 4 kDa, or between 7 kDa and 4 kDa. In some embodiments, the fraction comprises or consists of proteins of molecular weight between 15 kDa and 5 kDa, between 14 kDa and 5 kDa, between 13 kDa and 5 kDa, between 12 kDa and 5 kDa, between 11 kDa and 5 kDa, between 10 kDa and 5 kDa, between 9 kDa and 5 kDa, between 8 kDa and 5 kDa, or between 7 kDa and 5 kDa. In some embodiments, the fraction comprises or consists of proteins of molecular weight between 10 kDa and 2 kDa.

### Analysis of Low Molecular Weight Protein Fractions

Proteins fractionated may be examined by many methods including: bottom-up mass spectrometry; top-down mass spectrometry; structural analysis of individual components using X-ray crystallography or NMR; affinity-reagent based assays (for example with aptamers or antibodies) such as ELISA, western blotting, immunoprecipitation, or proximity extension assay.

In one embodiment, the protein fraction is analysed by bottom-up mass spectrometry as described below.

### Disulfide Bond Reduction, Alkylation and Trypsin Digestion

Proteins extracted using methods described above frequently contain multiple disulfide bonds, which may be reduced in the collected fraction obtained by SEC to avoid the introduction of intramolecular or intermolecular crosslinked peptides for downstream analysis by tandem mass spectrometry. In some embodiments, the reducing agent is TCEP. In some embodiments, the reducing agent is 2-mercaptoethanol. In some embodiments, the reducing agent is THPP. In some embodiments, the reducing agent is DTT.

In some embodiments, the reduced sulfhydryl groups are alkylated to prevent reformation of disulfide bonds. In some embodiments, the alkylating agent is iodoacetamide. In some embodiments, the alkylating agent is acrylamide. In some embodiments, the alkylating agent is N-ethylmaleimide. In some embodiments, the alkylating agent is 4-vinylpyridine. In some embodiments, the alkylating agent is iodoacetamide. In some embodiments, the alkylating agent is chloroacetamide. Disulfide bond reduction and alkylation are routine steps performed by the person skilled in the art to facilitate downstream identification of peptides during mass spectrometry, and many different reagents and conditions are commonly used (Suttapitugsakul et al. 2017 Molecular Biosystems 13(12):2574-2582).

In some embodiments, trypsin, or LysC, or a combination of trypsin and LysC is added to the samples to break down protein into fragments for downstream analysis. In some embodiments, trypsin is added at a ratio of 1:20 (µg trypsin to µg protein), or 1:50 (µg trypsin to µg protein), or 1:100 (µg trypsin to µg protein).

In some embodiments, a peptide clean-up step is performed using solid phase extraction following trypsin digestion and prior to downstream analysis. In some embodiments, peptides are bound to a strong cation exchange resin and eluted with a solution comprising ammonium hydroxide and acetonitrile.

In some embodiments, the sample is fractionated using reverse phase chromatography prior to downstream analysis. In some embodiments, high pH reverse phase chromatography is used. In some embodiments, peptides are resuspended in formic acid prior to high pH reverse phase chromatography. In some embodiments, buffers comprising acetonitrile and ammonium formate are used during fractionation.

### Mass Spectrometry

In some embodiments, samples are further analysed using mass spectrometry. In some embodiments, the mass spectrometer is a liquid chromatography-tandem mass spectrometer. In some embodiments, the mass spectrometer is a nano-liquid chromatography-tandem mass spectrometer. In some embodiments, the ionization technique is electrospray ionization and matrix-assisted laser desorption-ionization. In some preferred embodiments, the ionization technique is nanospray electrospray ionization. In some embodiments, peptides are resolved over a gradient from 5% acetonitrile to 40% acetonitrile on a reversed-phase chromatography column. In some embodiments, peptides are resolved over a gradient from 5% acetonitrile to 60% acetonitrile on a reversed-phase chromatography column. In some embodiments, peptides are resolved over a gradient from 5% acetonitrile to 90% acetonitrile on a reversed-phase chromatography column. In some embodiments, peptides are resolved over a gradient from 2% acetonitrile to 100% acetonitrile on a reversed-phase chromatography column. In some embodiments, fragmentation is achieved via collision-induced dissociation, higher-energy collisional dissociation, electron capture dissociation, or ultraviolet photodissociation. In some embodiments, fragmentation is achieved via higher energy collisional dissociation.

In some embodiments, the data acquisition method is data-dependent acquisition. In other embodiments, the data acquisition method is data-independent acquisition. In some other embodiments, the data acquisition method is selected reaction monitoring (SRM), also commonly known as multiple reaction monitoring (MRM). In some embodiments, the data acquisition method is parallel reaction monitoring (PRM). The types of analysis that can be performed using mass spectrometry are well known in the art (Zhang et al. 2014 Current protocols in molecular biology 108:10.21.1-10.21.30).

### Antibodies That Specifically Bind Erusiolin

In some embodiments, the present invention provides an antibody that specifically binds a peptide comprising or consisting of an amino acid sequence as defined in any one or more of SEQ ID NO. 1, SEQ ID NO. 2, SEQ ID NO. 3 and/or SEQ ID NO. 4. In further embodiments, an antibody is provided that specifically binds a peptide which may be described by the sequence X₁ X₂ X₃ X₄ X₅ X₆ X₇ X₈ X9 X₁₀ X₁₁ X₁₂ X₁₃ X₁₄ X₁₅ X₁₆ X₁₇, wherein:
X₁ is selected from A, L, V, and I, or is absent;
X₂ is selected from P and S, or is absent;
X₃ is selected from F and V;
X₄ is selected from L, M, V, I, A and F;
X₅ is selected from L, M, S, K, L, A and T;
X₆ is selected from E, Q, G and P;
X₇ is selected from D, Q, K and E;
X₈ is selected from P, S, E, A, Q and T;
X₉ is selected from A, S, D, T and V;
X₁₀ is selected from N and K;
X₁₁ is selected from Q, R, G, K and E;
X₁₂ is selected from F and I;
X₁₃ is selected from L, I and M;
X₁₄ is selected from R, H, G and Q;
X₁₅ is selected from L, M, H, F and Q;
X₁₆ is K or is absent; and
X₁₇ is R or is absent.

An antibody of the present invention may specifically bind a peptide with the sequence
A P F X₄ X₅ E X₇ X₈ A X₁₀X₁₁ F X₁₃ R L K R, or
A P F X₄ X₅ E X₇ X₈ A X₁₀ X₁₁ FX₁₃ R L, or
F X₄ X₅ E X₇ X₈ A X₁₀ X₁₁ F X₁₃ R L, or
F X₄ X₅ E X₇ X₈ A X₁₀ X₁₁ F X₁₃ R L K R, where:
X₄ is selected from L, M, V, I, A and F;
X₅ is selected from L, M, S, K, L, A and T;
X₇ is selected from D, Q, K and E;
X₈ is selected from P, S, E, A, Q and T;
X₁₀ is selected from N and K;
X₁₁ is selected from Q, R, G, K and E; and
X₁₃ is selected from L, I and M.

In other embodiments of the invention, the antibody may bind a peptide with the sequence:
A P F L X₅ E X₇ X₈ A N Q F X₁₃ R L K R, or
A P F L X₅ E X₇ X₈ A N Q F X₁₃ R L, or
F L X₅ E X₇ X₈ A N X₁₁ F Q R L, or
F L X₅ E X₇ X₈ A N X₁₁ F Q R L K R, where:
X₅ is selected from L, M, S, K, L, A and T;
X₇ is selected from D, Q, K and E;
X₈ is selected from P, S, E, A, Q and T.

Procedures for generating, purifying and modifying antibodies were originally developed during the 1970s and 1980s and have remained relatively unchanged since Harlow and Lane published “Antibodies: A Laboratory Manual” in 1988. The person skilled in the art would be familiar with techniques which may be used to produce antibodies using the disclosure of the epitope herein.

In some embodiments, the antibody of the present invention is a polyclonal antibody. The polyclonal antibody of the present invention may be a rabbit polyclonal antibody. In some embodiments, the antibody may be a mouse, rat, hamster, guinea pig, goat, sheep, horse or chicken polyclonal antibody. Any suitable animal may be used to produce the antibodies of the present invention. The choice of a suitable animal may be based on a number of factors, for example, the volume of antibodies required by the skilled addressee. The person skilled in the art will be aware that the rabbit is a popular choice for polyclonal antibody generation as an immune response to a wide range of small molecules may be easily elicited in a rabbit.

A range of immunisation techniques may be used to introduce the antigen to the animal. Non-limiting examples include injection into the skin or peritoneum. Subcutaneous or intramuscular injection may also be used to introduce the peptide antigen to the animal. A hapten may be used to elicit or enhance an immune response in order to produce the antibody. The hapten may be keyhole limpet hemocyanin (KLH). In some embodiments, ovalbumin may be used as a hapten. Any suitable hapten may be conjugated to the epitope to raise the antibodies of the present invention. An additional N-terminal cysteine may be added to the epitope to enable covalent coupling to the hapten.

In some embodiments, the antibody is a monoclonal antibody. Processes for the preparation of the monoclonal antibodies, derivatives and variants thereof, and antigen binding fragments thereof are readily available and capable of being performed without difficulty by persons of ordinary skill in the art. Monoclonal antibodies may be produced by the hybridoma method (see Kohler and Milstein,1975 Nature, 256:495-497; Coligan et al. section 2.5.1-2.6.7 in Methods In Molecular Biology (Humana Press 1992); and Harlow and Lane Antibodies: A Laboratory Manual, page 726 (Cold Spring Harbor Pub. 1988)), the EBV-hybridoma method for producing human monoclonal antibodies (see Cole, et al. 1985, in Monoclonal Antibodies and Cancer Therapy, Alan R. Liss, Inc., pp. 77-96), the human B-cell hybridoma technique (see Kozbor et al. 1983, Immunology Today 4:72), and the trioma technique.

In some exemplary embodiments, monoclonal antibodies may be prepared by administering the peptide antigen, for example, by intraperitoneal injection, to inbred or wild type mice (e.g. BALB/c or C57BL/6 mice), rabbits, rats, or other animal species, or transgenic mice capable of producing native or human antibodies. To induce an immune response, the immunogen may, for example, be mixed with an adjuvant, administered alone, expressed by a vector, administered as DNA, or administered as a fusion protein. The animal may be boosted, for example, at least twice, and spleen cells may then be harvested from the immunised animal. Hybridomas can be generated by fusing sensitised spleen cells with a myeloma cell line.

Monoclonal antibodies and antigen-binding fragments thereof may be recombinantly produced in any well-established expression system including, but not limited to, baculovirus, yeast (e.g. Pichia sp., Saccharomyces sp.), E. coli, mammalian cells, plants, or transgenic animals (see Breitling and Dubel, 1999, Recombinant Antibodies, John Wiley & Sons, Inc., NY, pp. 119-132).

### Uses of the Antibodies of the Present Invention

The antibodies of the present invention may be used in a number of techniques known to those skilled in the art. Non-limiting examples of applications of the antibodies include enzyme-linked immunosorbent assays (ELISA), mass spectrometry immunoassays (MSIA), Western blotting and immunoprecipitation. The antibodies of the present invention may be used in an ELISA to detect/and or quantify the peptides of the present invention in a biologic al sample. ELISAs comprise those based on colorimetry, chemiluminescence, and fluorometry. ELISAs have been successfully applied in the determination of low amounts of antigen in body tissues or fluids such as blood, serum, and plasma samples, and are well known in the art.

### Isolation and Characterisation of Isoforms of Erusiolin in a Sample

In some embodiments, erusiolin may be isolated from a biological sample. Isolation of erusiolin may be achieved by antibody-based immunoprecipitation using the antibodies provided by the present invention. The antibodies may be immobilised to a solid support such as magnetic particles or agarose resin. Additionally or alternatively, the antibodies may be free antibodies and may be allowed to form immune complexes with erusiolin prior to immobilisation. In some embodiments of the invention, immobilisation may be achieved by immunoglobulin (Ig)-binding proteins such as Protein A, Protein G, Protein A/G and Protein L. Antibodies may be bound to Ig-binding proteins using a crosslinker. In further embodiments, antibodies are immobilised to a solid support using streptavidin beads with biotinylated antibodies. In some embodiments, covalent antibody immobilisation strategies are used which chemically bind the antibody to the solid support. A wide variety of suitable immunoprecipitation techniques are well known in the art (see, for example, Bonifacino et al. 2016 Current protocols in molecular biology 71(1): 4.31.1-17.18.11).

Following immunoprecipitation, protein solutions may be characterised. In some embodiments of the present invention, protein solutions obtained by immunoprecipitation may be characterised by an unbiased analysis using liquid chromatography (LC) coupled to tandem mass spectrometry (MS/MS) without prior digestion. A wide variety of suitable techniques for the characterisation of protein solutions obtained by immunoprecipitation are well known in the art.

### Sources of Naturally Occurring Erusiolin

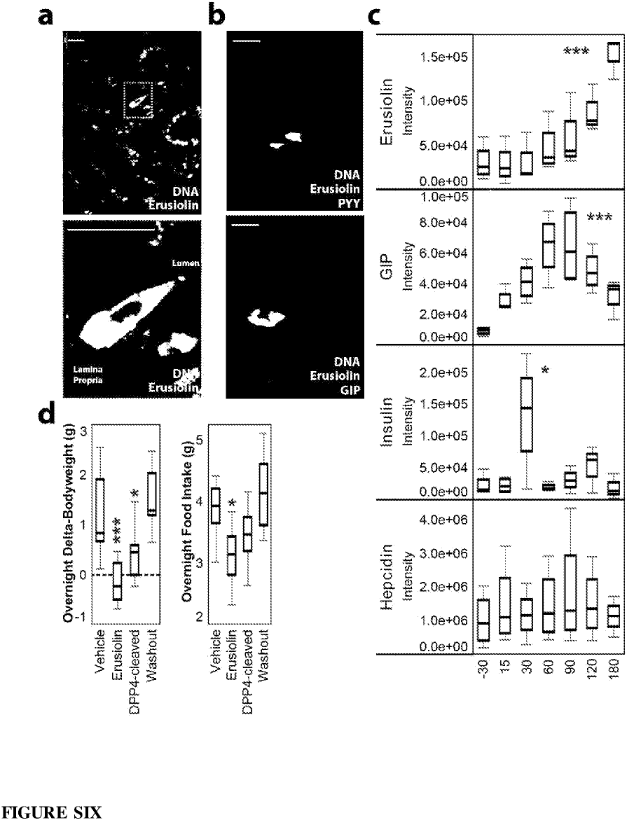

Erusiolin may be isolated from a biological sample from any biological source, such as an animal, a cell culture, or an organ culture. A non-limiting example of a method using a rabbit polyclonal antibody to identify erusiolin in human duodenum tissue is provided in FIG. 6. The erusiolin may be subsequently isolated from the samples and purified. Other non-limiting examples of biological samples which may be used to isolate the peptides of the present invention include plasma, serum, blood, cerebrospinal fluid, lymph, interstitial fluid, urine and saliva. In certain embodiments, samples are obtained from a mammal, such as a mouse, rat or primate. In some embodiments, mammalian animals are humans. The samples may be obtained from a person presenting in a clinical setting for diagnosis, prognosis or treatment of a disease or condition.

### Recombinant or Synthetic Erusiolin

Erusiolin is encoded by a conserved region of the human gene C3ORF85.In some embodiments of the present invention, erusiolin may be recombinant. In some embodiments, erusiolin may be produced by a polynucleotide encoding the “wild-type” or standard form of erusiolin. The polynucleotide may be operably linked to at least one heterologous regulatory element. The heterologous regulatory element may be a promoter or enhancer. In some embodiments, the polynucleotide may be introduced into a vector. In further embodiments, the nucleotide sequence of the polynucleotide may be modified to enhance expression in a heterologous host. Any number of modifications may be made to the nucleotide sequence to enhance expression. In some embodiments of the invention, several modifications of the same type may be made to the sequence. Additionally or alternatively, several different modifications may be made to the nucleotide sequence.

In some embodiments, the nucleotide sequence may be modified by codon optimisation. Organisms differ in their tendency to use specific codons over others to encode the same amino acid. Codon optimisation is a familiar technique to a person of ordinary skill in the art. Many publicly available online tools exist to enable the skilled artisan to optimise a nucleotide sequence or a protein sequence, for example http://genomes.urv.es/OPTIMIZER. In some embodiments of the present invention, the vector may be introduced into a host cell.

The polynucleotide sequence may have and/or encode more significant changes. One of skill in the art would appreciate that certain amino acids may be substituted for other amino acids within a protein without disruption of the biological activity of the protein. These altered sequences may not result in appreciable differences to, for example, the capacity of the protein to bind to structures such as binding sites on antibodies or cognate receptors. It is therefore contemplated by the present inventor that various so-called “conservative” changes may be made to the polynucleotides and/or proteins of the present invention whilst still fulfilling the goals of the invention.

Synthetic peptides may be used in the present invention. The peptides may be created by solid-phase peptide synthesis, which is an approach commonly used in the art (Paloma 2014 RSC Advances 4: 32658-32672). Additionally or alternatively, peptides may be ordered from one of the many suppliers or providers of custom peptide synthesis. These peptides may be stabilised by chemical modification to inhibit the cleavage of erusiolin due to endogenous human protease activity in tissues and plasma, including dipeptidyl peptidase 4 (DPP4) and other proteases. These synthetic peptides can be stabilised through various mechanisms, such as: changes in the amino acid sequence to retain function but decrease cleavage efficiency; N-terminal acetylation, or C-terminal amidation; N-methylation of the amide bind between amino acids such as between the 2^{nd} and 3^{rd} residue to inhibit DPP4 cleavage at this site; cyclisatio n to protect both termini and generate a rigid peptide backbone; or replacement of an L-stereoisomer amino acid residue with the equivalent D-stereoisomer such for the 2^{nd} residue to block DPP4 cleavage. In some embodiments of the invention, N-terminal acetylation protects a peptide from cleavage by a protease. In further embodiments, N-methylation protects a peptide bond from cleavage by a protease. In addition or alternatively, the replacement of L-amino acid stereoisomers with the equivalent D-form may protect connected peptide bonds from cleavage by a protease.

### Potential Applications of Erusiolin

Data presented in the present application has led the present inventor to postulate that erusiolin is a peptide hormone. Without being limited by theory, the high level of amino acid sequence conservation across species, the features of the conserved amino acid sequence shared with other peptide hormones (see Example Two), the detection of the peptide in blood plasma and the detection of the peptide in enteroendocrine-like cells of the gut, indicate that the peptides of the present invention have hormonal activity. Structural prediction data presented in the present application, indicating that the conserved amino acid sequence encodes structures similar to those of known secreted small protein hormones, also supports a hypothesis that erusiolin has hormonal activity (see Example Five).

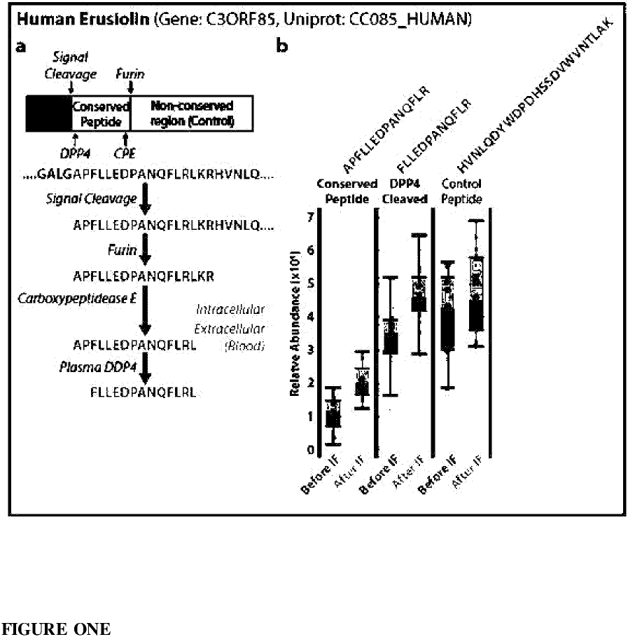

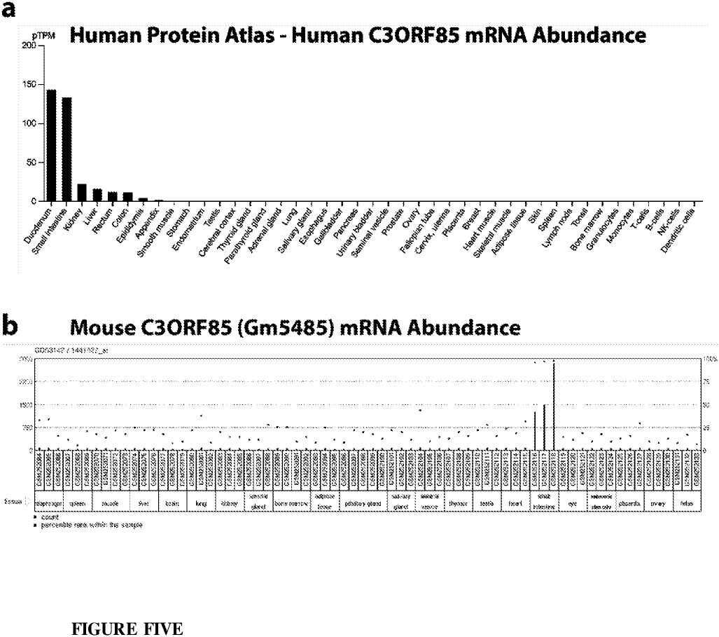

Erusiolin was increased in abundance in human plasma by an intermittent fasting diet where the human participants did not lose weight (FIG. 1b). It is shown that the abundance of erusiolin is induced by a mixed meal with equal portions of carbohydrates/proteins/lipids and that elevated levels during intermittent fasting may have been caused by the repeated large feeding bouts needed to maintain body weight. Data presented in Example One and Example Four of the present application indicates a potential role for the peptides of the present inventio n in appetite regulation. Without being limited by theory, the data has led to the hypothesis that the conserved peptide from erusiolin is secreted by the duodenum into blood plasma after food consumption and acts on the hypothalamus to activate transcription of the PWS locus, leading to satiety (fullness) signals (FIG. 5b).

The peptides of the present invention may be manipulated to regulate appetite. The peptides may be useful in suppressing appetite in subjects in need thereof, for example, subjects with type II diabetes or obesity. The peptides may be useful in stimulating appetite in subjects in need thereof, for example, subjects with cachexia or anorexia.

### EXAMPLES

The present invention will now be described with reference to the following specific examples, which should not be construed as in any way limiting.

### Example One: Detection of a Novel Peptide Hormone Which Was Significantly Increased in Abundance After 8-Weeks of Intermittent Fasting

In Australian provisional patent application number 2019901536, the entire disclosure of which is incorporated herein by cross-reference, the inventor of the present invention describes methods for the extraction and separation of proteins with a molecular weight under 15 kDa from a biological sample. The separated fraction may then be used for detection and quantitation of proteins in downstream analysis within a short timescale. These methods allow the identification and quantitation of peptides derived from active low abundance small-protein hormones in plasma. One protocol according to these methods dissociates protein-protein interactions, and removes large proteins > 10 kDa and small degradation products <2 kDa using size-exclusion chromatography prior to nanoLC-MS/MS analysis. This protocol was used during a human clinical trial testing the response to intermittent fasting (IF).

Intermittent fasting is a dietary intervention that is of significant interest due to its potential beneficial effects on metabolic health. However, very few studies have examined in an unbiased way the physiological changes induced by IF. In this example, the plasma hormone response was examined across a longitudinal clinical trial of IF in humans.

### 1.1 PREFER Trial and Inclusion Criteria

The PREFER randomized controlled trial was a discovery-based, single-centre study in Adelaide, South Australia and was registered with Clinicaltrials.gov (NCT01769976). The Royal Adelaide Hospital Research Ethics Committee approved the study protocol, and all participants provided written, informed consent prior to their inclusion. Each subject was assigned a number allowing for de-identification. A total of 88 women were enrolled in the study of which 25 were assigned to the intermittent fasting with weight maintenance group (IF100) analyzed in this example. Three of these participants withdrew during the diet period (2 due to time, 1 no longer wished to participate). The resulting 22 plasma samples plus 22 paired control samples were subjected to MS analysis with two samples from two different patients failing QC, leaving 40 patient samples for data analysis. Inclusion criteria for the study were: aged 35-70 years; BMI 25-42 kg/m²; weight-stable (within 5% of their screening weight) for >6 months prior to study entry; no diagnosis of type 1 or type 2 diabetes; non-smoker; sedentary or lightly active (i.e., <2 moderate to high-intensity exercise sessions per week); consumed < 140 g alcohol/week; no personal history of cardiovascular disease; no diagnosis of eating disorders or major psychiatric disorders (including those taking antidepressants); not pregnant or breastfeeding; and not taking medication that may affect study outcomes (e.g., phentermine, orlistat, metformin, excluding antihypertensive/lipid lowering medication). The active trial period was 10 weeks, comprised of a 2-week lead-in period, and 8 weeks of dietary intervention.

During the lead-in period, participants consumed their normal diet and maintained their weight. Following this, participants were placed on an IF diet at 100% of calculated baseline energy requirements per week (i.e., weight maintenance). Energy requirements were calculated using an average of two published equations, both of which use age, gender, height and weight variables. Due to the nature of the intervention, blinding was not possible. The Royal Prince Alfred Hospital Research Ethics Review Committee approved the study protocol (X17-0129 & HREC/17/RPAH/183), and all participants provided written, informed consent prior to their inclusion.

### 1.2 Diet

On fed days, participants were provided with food equal to -145% of energy requirements. On fasting days, participants consumed breakfast before 8 am (-37% of energy requirements were given at breakfast on fasting days) and were then instructed to “fast” for 24 h, until 8 am the following day. Participants were advised to fast on 3 non-consecutive weekdays per week. During the fasting period participants were allowed to consume water and limited amounts of energy-free foods (e.g., “diet” drinks, chewing gum, mints), black coffee and/or tea, and were provided with 250 mL of a very low energy broth (86 kJ/250 mL, 2.0 g protein, 0.1 g fat, 3.0 g carbohydrate) for either lunch or dinner. Participants were free-living, and foods were provided by fortnightly delivery to their home, except for fresh fruits and vegetables. Portions of fruits and vegetables were standardized and participants allowed to self-select according to the number of serves specified in their individual menus (~10% overall energy intake).

### 1.3 Blood Collection and Analysis

Fasted (10 h) plasma samples were obtained from participants in the IF100 group before and after the 8-week IF intervention. Blood samples were collected directly into purple K2-EDTA vacutainers (Becton Dickinson), placed on ice immediately after collection and spun <15 min post-collection at 1,500 g for 15 min at 4° C. The plasma samples were then frozen at -80° C. in cryotubes. Each sample was subject to <3 freeze-thaw cycles on ice.

### 1.4 Chemicals and Reagents

Acetonitrile (Optima grade), water (Optima grade), ammonia, formic acid and isopropanol (Optima grade) were from Thermo Fisher Scientific (Massachusetts, USA). Proteomics-grade trypsin (Catalogue number T6567) and all other reagents were from Sigma Aldrich (Missouri, USA).

### 1.5 Dissociation-Precipitation

Plasma samples were thawed on ice and vortexed prior to aliquoting 50 µL into a 2 mL tube (Eppendorf) at room temperature that contained 450 µL of extraction buffer (0.25 M HCl, 87.5% ethanol in H₂O). Samples were vortexed every 15 min and incubated for a total of 30 min at room temperature. Precipitated proteins were pelleted by centrifugation at 8,400 x g for 10 min at room temperature. The supernatant was moved to a new 2 mL tube and 125 µL of chloroform added and the tube vortexed. This was a key step to ensure lipid removal prior to SEC-based separations. To facilitate phase separation, 500 µL of water was added and the tube vortexed prior to centrifugation for 10 minutes at 7,000 x g at room temperature. The clear top phase (~850 µL) was retained (peptide supernatant) while avoiding any pellet and the yellow chloroform phase. The small-protein hormone containing supernatant was filtered using 0.45 µm Ultrafree®-MC HV centrifugal filter units (Merck Millipore - Massachusetts, USA) prior to moving the filtrate into vials for UHPLC-based SEC separation.

### 1.6 Denaturing Small-Protein Targeted SEC

To isolate proteins < 10 kDa from larger proteins in the small-protein hormone containing supernatant a Dionex Ultimate 3000 Bio-RS UHPLC system (Thermo Fisher Scientific) was used combined with an Agilent (California, USA) AdvanceBio SEC column with 130 Å pores, 2.7 µm particles, dimensions of 7.8 x 300 mm. The column was equilibrated with 10 column volumes of denaturing SEC running buffer (30% acetonitrile and 0.1% TFA) prior to sample analysis. Standards consisting of either ubiquitin (Sigma - 79586-22-4), or a HPLC peptide standard mix (Sigma - H2016-1VL) diluted in SEC running buffer were used for mass calibration. The small-protein hormone containing supernatant was stored in the auto-sampler at 4° C. prior to analysis and for each SEC separation, 200 µL was injected onto the column. Each SEC separation was performed for 25 min (1.5 column volumes) at a flow rate of 1 mL min⁻¹ at a column temperature of 30° C. The eluting proteins were monitored by UV absorbance at 215 and 280 nm. Only one fraction was collected into a low protein binding 2 mL tube or 2 mL 96-deep-well plate (Eppendorf) between 6 min to 8 min retention time. This fraction corresponded to proteins of molecular weight between < 10 kDa and >2 kDa, which had a total volume of 2 mL, and the fractions were stored at 4° C. prior to subsequent processing.

### 1.7 Peptide Reduction, Alkylation and Trypsin Digestion

The collected fraction was dried using a GeneVac EZ-2, using the HPLC setting at 45° C. and the dried proteins were resuspended in 100 µL of 50 mM triethylammonium bicarbonate (TEAB) pH 8.5 in H₂O. Disulfide bonds were reduced by addition of DTT to a final concentration of 5 mM and incubated on a thermomixer at 95° C. at 1000 rpm for 10 min. To alkylate the reduced sulfhydryl groups chloroacetamide was added to a final concentration of 20 mM and samples incubated on a thermomixer at 95° C. at 1000 rpm for 10 min. For trypsin digestion samples were cooled to room temperature and trypsin was added at a ratio of 1:20 (trypsin to protein), where each SEC fraction contained ~ 4 µg protein and therefore 200 ng was added. The samples were incubated for 16 h at 37° C. at 500 rpm on a thermomixer to digest. To stop the digest 10% TFA in H₂O was added to achieve a 1 % final concentration.

### 1.8 Peptide Clean-Up Using SDB-RPS StageTips

SDB-RPS StageTips were generated by punching double-stacked SDB-RPS discs (Sigma, Cat#66886-U) with an 18-gauge needle and mounted in 200 µL tips (Eppendorf). For clean-up utilizing the Spin96, StageTips were inserted into a holder and placed in the top, which was then stacked onto the wash-bottom containing a polypropylene 96-well microtitre plate to collect the sample flow-through and washes. Each tip was wetted with 100 µL of 100% acetonitrile and centrifuged at 1,000 x g for 1 min. Following wetting, each StageTip was equilibrated with 30% methanol/1% TFA, followed by 100 µL of 0.1% TFA in H₂O, with centrifugation for each at 1,000 x g for 3 min. Each StageTip was then loaded with the equivalent of ~10 µg peptide in 1 % TFA (< 100 µL total volume per spin). The peptides were washed once with 100 µL of 0.2% TFA in water, which was followed by one wash with 100 µL of 99% isopropanol/1% TFA. For elution of peptides, the wash- bottom was exchanged with a bottom containing a holder supporting an unskirted PCR plate that has been trimmed to fit. To elute, 100 µL of 5 % ammonium hydroxide/80% acetonitrile was added to each tip and centrifuged as above for 5 min. Samples in the PCR plate were dried using a GeneVac EZ-2 using the ammonia setting at 35° C. for 1 h 15 m. Dried peptides were resuspended in 7 µL of 5% formic acid per 200 µL SEC injection of small-protein hormone-containing supernatant and stored at 4° C. until analyzed by LC-MS.

### 1.9 High pH Reverse Phase Chromatography

Pooled trypsin digested plasma proteins (100 µg total) in 5% formic acid were subjected to high pH reverse phase chromatography on a Thermo Scientific Dionex Ultimate 3000 BioRS system with a fractionation auto-sampler, using a Waters XBridge Peptide BEH C18 column (130 Å, 3.5 µm, 4.6 mm X 250 mm, Cat No. 186003570). The column was incubated at 30° C. with a constant flow rate of 1 mL/min, with buffer A containing 2% acetonitrile (ACN) and 10 mM ammonium formate (pH 9) and buffer B containing 80% ACN and 10 mM ammonium formate (pH 9). Fractions were collected every 8.75 s from a retention time of 2 min to 16 min (96 pseudo fractions, concatenated into 16 fractions total). Peptides were separated by a linear gradient from 10% to 40% buffer B for the first 11 min and 100% buffer B for the remaining time. The fractions were collected in a 2 mL protein low-bind 96-well deepwell plate (Eppendorf) across 16 wells in a concatenated pattern using tube wrapping.

### 1.10 DIA

Data-independent acquisitions were performed using variable isolation widths for different m/z ranges. Stepped normalized collision energy of 25 +/- 10% was used for all DIA spectral acquisitions.

### 1.11 LC-MS/MS Data Analysis - Spectronaut Pulsar X directDIA Analysis

RAW data were analyzed using the quantitative proteomics software Spectronaut Pulsar X (version 12.0.20491.11.25225 (Jocelyn)). The database supplied to the search engine for peptide identifications was a focused database generated from an earlier fractionated plasma small-protein hormone analysis. Enzyme specificity was set to semi-specific N-ragged trypsin (cleavage C-terminal to Lys and Arg) with a maximum of 2 missed cleavages permitted. Deamidation of Asn and Gln, oxidation of Met, pyro-Glu (with peptide N-term Gln) and protein N-terminal acetylation were set as variable modifications. Carbamidomethyl on Cys was searched as a fixed modification. The workflow was set to use iRT profiling. The FDR was set to 1% using a target-decoy approach. Spectronaut generated a custom mass tolerance for each precursor ion. The threshold for accepting a precursor was set at a Q value <0.01 and each precursor must have >3 fragment ions. All other settings were factory default. Processed data was analyzed and statistical tests performed using the R software package (version 3.4.3) with plot generated using Tableau (version 10.0.2).

### 1.12 PREFER Trial Statistical Analysis

This example provides an analysis of the intermittent fasting with weight maintenance group (IF100), with plasma samples collected before and after an 8-week period of intermittent fasting. A Wilcox robust test was applied to these data to allow for proteins whose distributio n for the difference between treatment groups across participants was not normally distributed. Specifically, Yuen’s test was used on trimmed means for dependent (paired) samples. Fold changes comparing plasma peptide precursor abundance before and after intermittent fasting were calculated using the median. For plotting the variation in individual peptide precursor intensity measurements and clinical measures in response to IF, adjusted values were calculated that forced all participants to have the same mean value for each measure. For all datasets statistical analyses were performed using R (version 3.4.3) and processed data was plotted using Tableau (version 10.0.2). Data are shown as median ± 95% confidence interval, unless otherwise stated. Significance was set at P<0.05.

### 1.13 Results

The analysis quantified >3400 peptide precursors and peptides from a wide variety of active hormones/factors were identified, including IGF-1, insulin, GLP-1, GIP, ghrelin, hepcidin, IL-36gamma, RANTES, chromogranin, SDF-1, granulins, chemokines, defensins and guanylin. Using a paired test statistic, 235 peptide precursors were detected that were significantly changed (P<0.05) due to the IF intervention. Two tryptic peptides (APFLLEDPANQFLR & FLLEDPANQFLR) derived from the conserved region of the uncharacterised 8 kDa protein encoded by the human gene C3ORF85 and one peptide derived from the non-conserved region were detected. Excitingly, the two highly conserved peptides from C3ORF85 were significantly increased in abundance after 8 weeks of intermittent fasting (FIG. 1b). From this, it was hypothesised that the peptides were derived from a peptide hormone, the abundance of which was induced by intermittent fasting diet, and that elevated levels were caused by the repeated large feeding bouts used in the trial to maintain body weight.

### Example Two: The Protein Sequence of the Novel Peptide Hormone Is Conserved to Jawed Fish

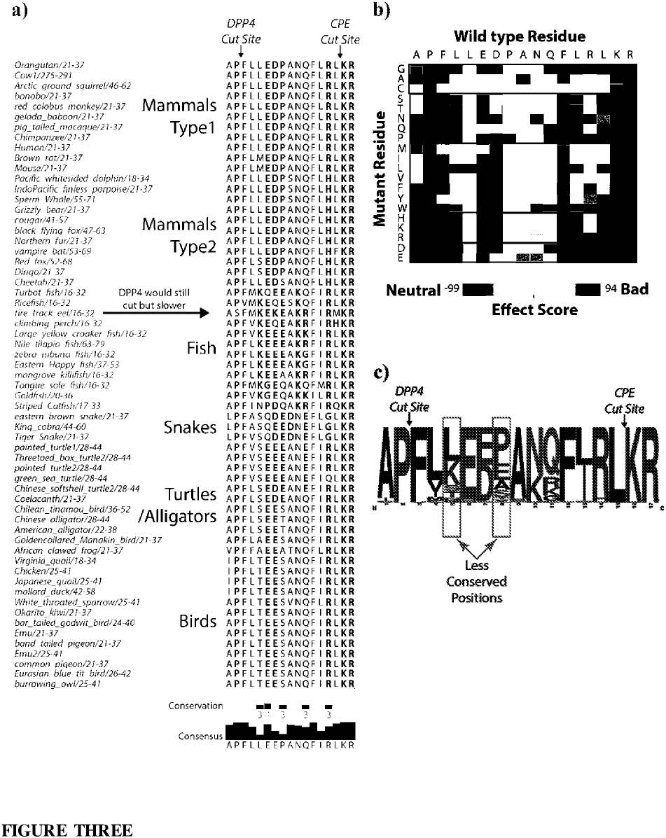

Multiple sequence alignment showed that the protein sequence of the novel peptide hormone is conserved to jawed fish and the N-terminus of the protein (after the signal peptide, which targets the protein for secretion) shows high protein sequence conservation up to a high-scoring furin cleavage site (FIGS. 1 to 3). Furin is a ubiquitous endoprotease within the secretory pathway that cleaves C-terminal to the R-X-(K/R)-R motif found in many small protein hormones. After furin cleavage, the conserved erusiolin peptide is likely a substrate for carboxypeptidase E (CPE), which removes dibasic residues (arginine/lysine) from the C-terminus of many peptide hormones. The conserved erusiolin peptide has a proline in position 2, which conforms to the consensus motif for dipeptidyl peptidase 4 (DPP4), which is a common processing enzyme for hormones to induce either their degradation (e.g. GLP-1), or their activation (e.g. neuropeptide Y - NPY).

### Example Three: Isolation and Characterisation of the Most Abundant Forms of the Novel Peptide Hormone

### 3.1 Generation of an Erusiolin-Specific Rabbit Polyclonal Antibody

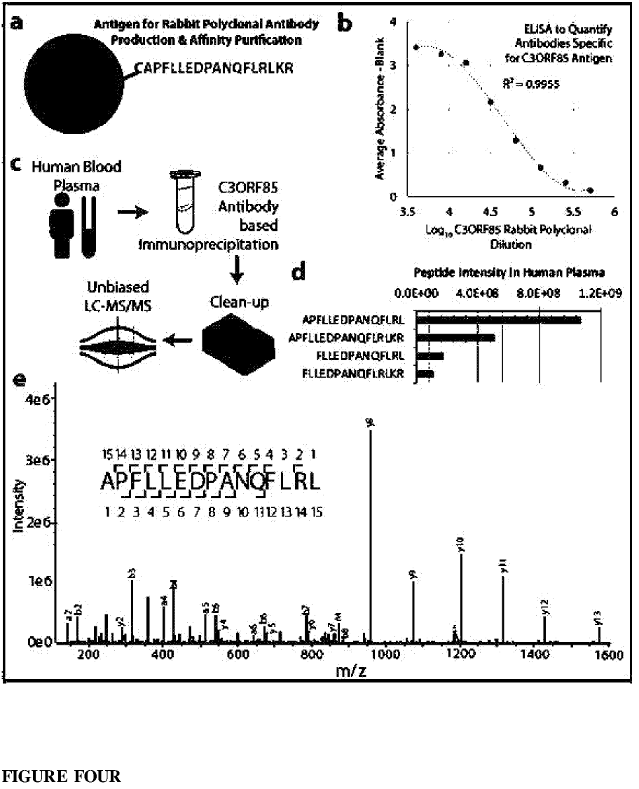

A rabbit polyclonal antibody was generated using a commercial supplier (Genscript) by immunisation of 2 rabbits with a synthetic form of the full-length human C3ORF85 conserved sequence. An additional N-terminal cysteine was added to the sequence to enable covalent coupling to keyhole limpet hemocyanin (KLH) for increased immunogenicity (CAPFLLEDPANQFLRLKR) (FIG. 4a). After 4 immunisations with this peptide-LKH conjugate, the rabbits were exsanguinated and antibodies in the serum were affinity purified. Affinity purified antibodies were stored at 1 mg/ml in PBS containing 0.02 % sodium azide at 4° C.

### 3.2 Mass Spectrometry Immunoassay (MSIA) From Human Plasma

Human blood plasma (EDTA, with addition at the time of collection of benzamidine-HCl and vildagliptin to final concentrations of 50 mM and 100 nM, respectively) stored at -80° C. was thawed on ice for 1 h. Plasma (100 uL) was mixed with an equal volume of dilution buffer (100 mM beta-octyl-D-glucopyranoside in PBS) on ice and vortexed briefly. The diluted plasma was clarified by centrifugation at 18,000 g for 10 min at 4° C. and the supernatant moved to a new 1.5 mL tube. Thermo Pierce Protein G magnetic beads (20 uL of slurry) was washed in PBS and resuspended in 250 uL of PBS. Either a negative control antibody (total rabbit IgG), or the erusiolin-specific rabbit polyclonal antibody was added (1 uL of 1 mg/mL stock) to the Protein G beads in PBS and incubated for 30 min at RT with rotation. The beads were washed once in 250 uL of dilution buffer and then once in 1000 uL of PBS, prior to addition of 1000 uL of Crosslinking Buffer (250 mM disuccinimidyl suberate in PBS) and incubation for 30 min at RT with rotation. The beads were quenched with 1000 uL of 0.1 M Glycine-HCl pH 2.8 and incubation for 5 min at RT. Crosslinked beads were then washed in 1000 uL of PBS, prior to addition of the 200 uL of diluted plasma and incubation for 2 h at 4° C. with rotation. The beads were washed a total of 2 times in 1 mL per wash of wash buffer (PBS) at RT. The beads were washed a total of 2 times in 1 mL per wash of LC-MS grade water at RT. The immunoprecipitated peptides were eluted in 250 uL of elution buffer (30% acetonitrile in aqueous 0.1 % TFA, LC-MS grade), for 10 min at RT, vortexing occasionally.

### 3.3 Peptide Clean-Up Using C18 StageTips

C18 StageTips were generated by punching double-stacked C18 discs (Sigma) with an 18-gauge needle and mounted in 200 µL tips (Eppendorf). For clean-up utilizing the Spin96, StageTips were inserted into a holder and placed in the top, which was then stacked onto the wash-bottom containing a polypropylene 96-well microtitre plate to collect the sample flow-through and washes. Each tip was wetted with 100 µL of 100% acetonitrile and centrifuged at 1,000 x g for 1 min. Following wetting, each StageTip was equilibrated with 100 µL of 0.1% TFA in H₂O, with centrifugation for each at 1,000 x g for 3 min. Each StageTip was then loaded with the plasma IP eluate (<100 µL total volume per spin). The peptides were washed twice with 100 µL of 0.1% TFA in water. For elution of peptides, the wash- bottom was exchanged with a bottom containing a holder supporting an unskirted PCR plate that has been trimmed to fit. To elute, 100 µL of 50% acetonitrile 0.1% TFA in H₂O was added to each tip and centrifuged as above for 5 min. Samples in the PCR plate were dried using a GeneVac EZ-2 using the HPLC setting at 35° C. for 1 h 15 m. Dried peptides were resuspended in 10 µL of 5% formic acid per IP and stored at 4° C. until analyzed by LC-MS.

### 3.4 LC-MS/MS Data Acquisition and Analysis

Using a Thermo Fisher Dionex RSLCnano uHPLC, peptides in 5% (v/v) formic acid (injection volume 3 µL) were directly injected onto a 45 cm x 75 µm C18 (Dr. Maisch, Ammerbuch, Germany, 1.9 µm) fused silica analytical column with a ~10 µm pulled tip, coupled online to a nanospray ESI source. Peptides were resolved over gradient from 5% acetonitrile to 40% acetonitrile over various gradient lengths ranging from 15 min to 140 min with a flow rate of 300 nL min⁻¹. Peptides were ionized by electrospray ionization at 2.3 kV. Tandem mass spectrometry analysis was carried out on either a Q-Exactive HF, or HFX mass spectrometer (ThermoFisher) using HCD fragmentation. The data-dependent acquisition method used acquired MS/MS spectra of the top 10 most abundant ions at any one point during the gradient. RAW data were analysed using the quantitative proteomics software MaxQuant(version 1.5.7.0). This version of MaxQuant includes an integrated search engine, Andromeda. Peptide and protein level identification were both set to a false discovery rate of 1% using a target-decoy based strategy. The database supplied to the search engine for peptide identifications contained both the human UniProt database and the MaxQuant contaminants database. Mass tolerance was set to 4.5 ppm for precursor ions and MS/MS mass tolerance was 20 ppm. Enzyme specificity was set to semi-specific N-ragged trypsin (cleavage C-terminal to Lys and Arg) with a maximum of 2 missed cleavages permitted for the main search and fully specific trypsin (cleavage C-terminal to Lys and Arg) for the first search. Deamidation of Asn and Gln, oxidation of Met, pyro-Glu (with peptide N-term Gln) and protein N-terminal acetylation were set as variable modifications. Maxquant output was processed and statistical tests performed using the R software package (version 3.4.3). Processed data was plotted using Tableau (version 10.0.2).

### 3.5 Results

The erusiolin-specific rabbit polyclonal antibody was tested for performance and titre using an enzyme-linked immunosorbent assay (ELISA) (FIG. 4b). The peptide antigen was coated onto ELISA plates prior to addition of the antibody at various dilutions indicated on the x-axis. The corresponding absorbance signal shown on the y-axis indicates antibody binding. Antibody binding signal (>0.5) was observed down to dilutions of 1:128,000 indicating the antibody has a high titre. The antibody was subsequently used for a mass spectrometry immunoassay (MSIA) from human plasma (FIG. 4c), which enabled the isolation of all of the circulating forms of erusiolin peptides. These were subsequently characterised in the undigested state using unbiased top-down analysis by liquid chromatography (LC) coupled to tandem mass spectrometry (MS/MS). This showed that the most abundant form of erusiolin in human plasma was APFLLEDPANQFLRL (FIGS. 4d and 4e).

### Example Four: The mRNA Encoding the Erusiolin Protein is Largely Duodenum-Specific (Proximal Small Intestine) in Both Humans and Mice and the Protein is Expressed in Rare Cells of the Mucosal Layer Consistent With Enteroendocrine Cells These Cells Secrete Erusiolin Into the Blood After a Meal and Injection of Elevated Levels of Erusiolin Lead to an Inhibition of Food Intake in Mice

### 4.1 Formaldehyde Fixation and Cryosectioning of Duodenum Tissue Samples

Small pieces (<5 mm in any dimension) of duodenum from humans were fixed at room temperature for 2 h in 20 mL of 4% formaldehyde (Methanol-free, EM-grade, Thermo, Fisher) diluted in PBS on a rocker. The tissue was then immersed in 20 mL of 30% sucrose in PBS at 4° C. overnight or until tissue has sunk. Tissue was placed into Tissue Tek Cryomoulds (15mm x 15mm x 5mm for mouse duodenum) and all tissue covered in OCT. The mould containing the tissue block was slowly placed into liquid nitrogen to freeze. The frozen tissue block was stored at -80° C. until sectioning. The frozen tissue block was placed into a cryostat (-20° C.) prior to sectioning and the temperature of the frozen tissue block allowed to equilibrate. The frozen tissue block was sectioned into a desired thickness (10 µm) The tissue sections were placed onto gelatin-coated glass slides suitable for immunohistochemistry. Sections were stored in a sealed slide box at -80° C.

### 4.2 Slide Section Antigen Retrieval and Antibody Staining

Air dried slides (60 min at RT) were washed three times for 5 min each with PBS at RT. Slides were incubated in 1 x DAKO antigen retrieval solution for 20 min at 95° C. Slides were then incubated in fresh 1 x DAKO antigen retrieval solution for 20 min at RT. PAP rings were drawn around tissue sections without letting the tissue section dry. Slides were washed three times for 5 min each with PBSTx (0.2% Triton X-100 in PBS). Slides were blocked for 60 min at RT with 10% donkey sera in PBSTx. Slides were then washed three times for 2 min each with PBSTx. The slides were then incubated with anti-erusiolin antibody diluted 1:1000 in PBSTx overnight at 4° C. Slides were washed three times for 5 min each with PBSTx. The slides were then incubated with Secondary Antibodies (donkey anti-rabbit AF488) diluted 1:200 in PBSTx for 60 min at RT. Slides were then washed three times for 5 min each with PBSTx. Slides were mounted with either ProLong Antifade Gold plus DAPI and stored at RT.

### 4.3 Mixed Meal Test in Healthy Humans

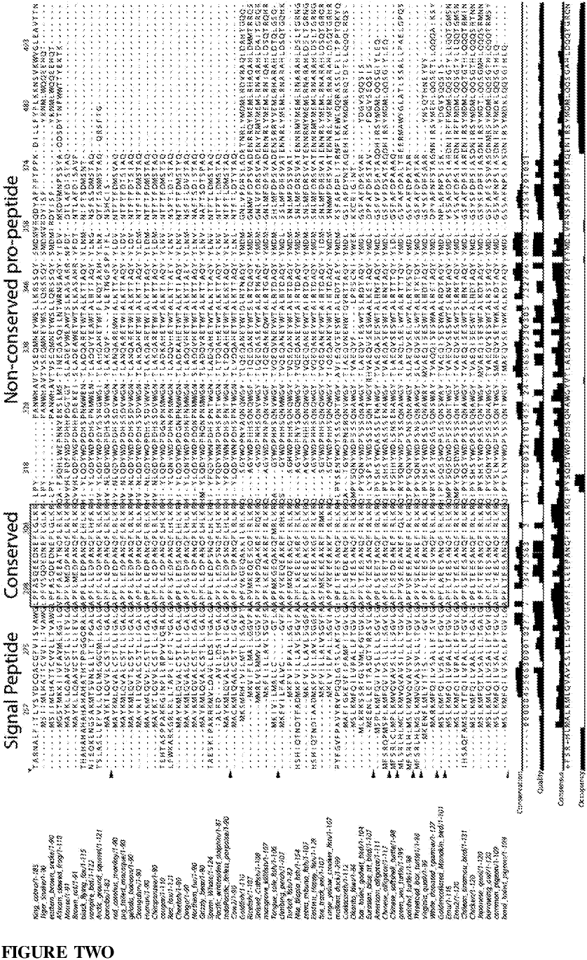

The study was conducted according to the principles outlined in the declaration of Helsinki and the study protocol was approved by the St Vincent’s Hospital Human Research Ethics Committee (Sydney, Australia). All participants provided written informed consent prior to study commencement. The study was registered at clinicaltrials.gov (NCT02501343). Participants were asked not to exercise or drink alcohol for 48 h prior to the meal studies. To minimize variation between studies, participants were asked to follow a similar routine before the studies, with particular attention to the meal content on the night before the study. Participants reported to the Clinical Research Facility in the morning after an overnight fast, placed on a hospital bed, and an 18-gauge intravenous cannula inserted in the antecubital fossa. Baseline venous blood samples were ascertained at t = -30 min before administration of the intervention (FIG. 2). The meal consisted of two breakfast muffins and 250 mL apple juice. Participants were given 20 min to consume the meal, with the 3 h follow up commencing at the completion of the meal (time = 0 min). Participants remained in bed in a reclined position for the following 3 h, with blood samples drawn periodically. Blood samples were collected directly into purple K2-EDTA vacutainers with dipeptidyl peptidase-4 inhibitor (vildagliptin) and trasylol to minimize peptide hormone degradation after collection, placed on ice immediately after collection and spun <15 min post-collection at 1,500 g for 15 min at 4° C. The plasma samples were then frozen at -80° C. in cryotubes. Each sample was subject to <3 freeze-thaw cycles on ice.

### 4.4 Chemicals and Reagents

Acetonitrile (Optima grade), water (Optima grade), ammonia, formic acid and isopropanol (Optima grade) were from Thermo Fisher Scientific (Massachusetts, USA). Proteomics-grade trypsin and all other reagents were from Sigma Aldrich (Missouri, USA).

### 4.5 Dissociation-Precipitation

Plasma samples were thawed on ice and vortexed prior to aliquoting 50 µL into a 2 mL tube (Eppendorf) at room temperature that contained 450 µL of extraction buffer (0.25 M HCl, 87.5% ethanol in H₂O). Samples were vortexed every 15 min and incubated for a total of 30 min at room temperature. Precipitated proteins were pelleted by centrifugation at 8,400 x g for 10 min at room temperature. The supernatant was moved to a new 2 mL tube and 125 µL of chloroform added and the tube vortexed. This was a key step to ensure lipid removal prior to SEC-based separations. To facilitate phase separation, 500 µL of water was added and the tube vortexed prior to centrifugation for 10 minutes at 7,000 x g at room temperature. The clear top phase (~850 µL) was retained (peptide supernatant) while avoiding any pellet and the yellow chloroform phase. The small-protein hormone containing supernatant was filtered using 0.45 µm centrifugal filter units prior to moving the filtrate into vials for UHPLC-based SEC separation.

### 4.6 Denaturing Small-Protein Targeted SEC

To isolate proteins < 10 kDa from larger proteins in the small-protein hormone containing supernatant a Dionex Ultimate 3000 Bio-RS UHPLC system (Thermo Fisher Scientific) was used combined with an Agilent AdvanceBio SEC column with 130 Å pores, 2.7 µm particles, dimensions of 7.8 x 300 mm. The column was equilibrated with 10 column volumes of denaturing SEC running buffer (30% acetonitrile and 0.1% TFA) prior to sample analysis. Standards consisting of either ubiquitin or a HPLC peptide standard mix diluted in SEC running buffer were used for mass calibration. The small-protein hormone containing supernatant was stored in the auto-sampler at 4° C. prior to analysis and for each SEC separation, 200 µL was injected onto the column. Each SEC separation was performed for 25 min (1.5 column volumes) at a flow rate of 1 mL min⁻¹ at a column temperature of 30° C. The eluting proteins were monitored by UV absorbance at 215 and 280 nm. Only one fraction was collected into a low protein binding 2 mL tube or 2 mL 96-deep-well plate between 6 min to 8 min retention time. This fraction corresponded to proteins of molecular weight between < 10 kDa and >2 kDa, which had a total volume of 2 mL, and the fractions were stored at 4° C. prior to subsequent processing.

### 4.7 Peptide Reduction, Alkylation and Trypsin Digestion

The collected fraction was dried using a GeneVac EZ-2, using the HPLC setting at 45° C. and the dried proteins were resuspended in 100 µL of 50 mM triethylammonium bicarbonate (TEAB) pH 8.5 in H₂O. Disulfide bonds were reduced by addition of DTT to a final concentration of 5 mM and incubated on a thermomixer at 95° C. at 1000 rpm for 10 min. To alkylate the reduced sulfhydryl groups chloroacetamide was added to a final concentration of 20 mM and samples incubated on a thermomixer at 95° C. at 1000 rpm for 10 min. For trypsin digestion samples were cooled to room temperature and trypsin was added at a ratio of 1:20 (trypsin to protein), where each SEC fraction contained ~ 4 µg protein and therefore 200 ng was added. The samples were incubated for 16 h at 37° C. at 500 rpm on a thermomixer to digest. To stop the digest 10% TFA in H₂O was added to achieve a 1% final concentration.

### 4.8 Peptide Clean-Up Using SDB-RPS StageTips

SDB-RPS StageTips were generated by punching double-stacked SDB-RPS discs (Sigma, Cat#66886-U) with an 18-gauge needle and mounted in 200 µL tips. For clean-up utilizing the Spin96, StageTips were inserted into a holder and placed in the top, which was then stacked onto the wash-bottom containing a polypropylene 96-well microtitre plate to collect the sample flow-through and washes. Each tip was wetted with 100 µL of 100% acetonitrile and centrifuged at 1,000 x g for 1 min. Following wetting, each StageTip was equilibrated with 30% methanol/1% TFA, followed by 100 µL of 0.1% TFA in H₂O, with centrifugation for each at 1,000 x g for 3 min. Each StageTip was then loaded with the equivalent of ~10 µg peptide in 1% TFA (< 100 µL total volume per spin). The peptides were washed once with 100 µL of 0.2% TFA in water, which was followed by one wash with 100 µL of 99% isopropanol/1% TFA. For elution of peptides, the wash- bottom was exchanged with a bottom containing a holder supporting an unskirted PCR plate that has been trimmed to fit. To elute, 100 µL of 5 % ammonium hydroxide/80% acetonitrile was added to each tip and centrifuged as above for 5 min. Samples in the PCR plate were dried using a GeneVac EZ-2 using the ammonia setting at 35° C. for 1 h 15 m. Dried peptides were resuspended in 7 µL of 5% formic acid per 200 µL SEC injection of small-protein hormone-containing supernatant and stored at 4° C. until analyzed by LC-MS.

### 4.9 DIA

Data-independent acquisitions were performed using variable isolation widths for different m/z ranges. Stepped normalized collision energy of 25 +/- 10% was used for all DIA spectral acquisitions.

### 4.10 LC-MS/MS Data Analysis - Spectronaut Pulsar X directDIA Analysis

RAW data were analyzed using the quantitative proteomics software Spectronaut Pulsar X. The database supplied to the search engine for peptide identifications was a focused database generated from an earlier fractionated plasma small-protein hormone analysis. Enzyme specificity was set to semi-specific N-ragged trypsin (cleavage C-terminal to Lys and Arg) with a maximum of 2 missed cleavages permitted. Deamidation of Asn and Gln, oxidation of Met, pyro-Glu (with peptide N-term Gln) and protein N-terminal acetylation were set as variable modifications. Carbamidomethyl on Cys was searched as a fixed modification. The workflow was set to use iRT profiling. The FDR was set to 1% using a target-decoy approach. Spectronaut generated a custom mass tolerance for each precursor ion. The threshold for accepting a precursor was set at a Q value <0.01 and each precursor must have >3 fragment ions. All other settings were factory default.

### 4.11 Mixed Meal Test Statistical Analysis

A repeated-measures one-way ANOVA was used to determine significance in the response at any timepoint. Correction for multiple testing was performed using the method of Benjamini-Hochberg. Statistical analyses were performed using R (version 3.4.3) and processed data was plotted using Tableau (version 10.0.2). Data are shown as median ± 95% confidence interval, unless otherwise stated. Significance was set at P<0.05.

### 4.12 Erusiolin Peptide Synthesis and TFA Removal

In these experiments “erusiolin” refers to the synthetic peptide of sequence APFLLEDPANQFLRL (SEQ ID NO: 2), “DPP4-cleaved” refers to the synthetic peptide of sequence FLLEDPANQFLRL (SEQ ID NO: 3). These peptides were synthesised and purified as the TFA-salt by a commercial supplier (Genscript), which generated 10 mg of each peptide at >90% purity. TFA removal and formation of the HCL-peptide salt was performed by freeze-drying the peptide solution (in LC-grade H₂O) for 16 h and resuspension in 500 uL of 10 mM HC1 in LC-grade H₂O. This solution was then freeze-dried for 16 h. A total of three rounds of 10 mM HC1 resuspension and freeze-drying for 16 h were performed. After the final drying step, the peptides were resuspended in LC-grade H₂O. The peptide solutions were analysed for TFA contamination and determination of the peptide concentration using FT-IR spectroscopy. No TFA contamination was observed and the final peptide concentrations were 5 mg/mL in LC-grade H₂O. Peptides were diluted in vehicle (1 mg/ml mouse albumin in PBS) before injection.

### 4.13 Crossover Peptide Injection Trial in Mice

C57BL/6J male mice at 14 weeks of age were treated with either vehicle, erusiolin, or the DPP4-cleaved peptide in a crossover trial study using intraperitoneal injection of the peptides. Each mouse was randomly allocated to a sequence of treatments. Each sequence had treatments in a random order. One treatment was performed per mouse per day and injections were at 5pm just before the night feeding period. Between each treatment day a washout day was included where no injection was performed to allow the previous days treatment to be degraded and minimise confounding effects. The food intake and body weight of each animal was measured just prior to injection and again 16 h after injection. This enable the calculation of the mass delta (change) for food intake and body mass. A repeated-measures one-way ANOVA was used to determine significance between the vehicle negative control treatment and the other treatments. Statistical analyses were performed using R (version 3.4.3) and processed data was plotted using Tableau (version 10.0.2). Data are shown as median ± 95% confidence interval, unless otherwise stated. Significance was set at P<0.05.

### 4.14 Results

The mRNA encoding the erusiolin protein is largely duodenum-specific (proximal small intestine) in both humans (FIG. 5a) and mice (FIG. 5b). Using our erusiolin-specific antibody we have used immunohistochemistry to confirm that there are erusiolin-positive cells in the duodenum of humans and mice and that these cells are rare, the staining is cytoplasmic and is therefore consistent with an enteroendocrine cell type (FIG. 6a). Furthermore, erusiolin staining overlapped in cells that were also positive for the enteroendocrine cell marker GIP (FIG. 6b). Analysis of peptide hormones in human plasma before and after a mixed meal test in humans showed that erusiolin was significantly increased in abundance starting 1 h after the meal and continued to increase for up to 3 h post-meal (FIG. 6c). This response was distinct from insulin and GIP.

In this example, we tested the effect of supraphysiological intraperitoneal injection of erusiolin on acute food intake we conducted a randomised crossover trial analysis of food intake and body weight responses in mice for 16h after injection. This crossover design minimised animal numbers required and corrected for confounding factors between animals. We used synthetic peptides corresponding to the core erusiolin sequence APFLLEDPANQFLRL (SEQ ID NO: 2) and the DPP4-cleaved form of this peptide FLLEDPANQFLRL (SEQ ID NO: 3). The core erusiolin sequence showed a significant decrease in food intake and body weight compared to vehicle injection, DPP4-cleaved peptide injection, and the intervening washout days (between treatments), which were not significant or less significant.

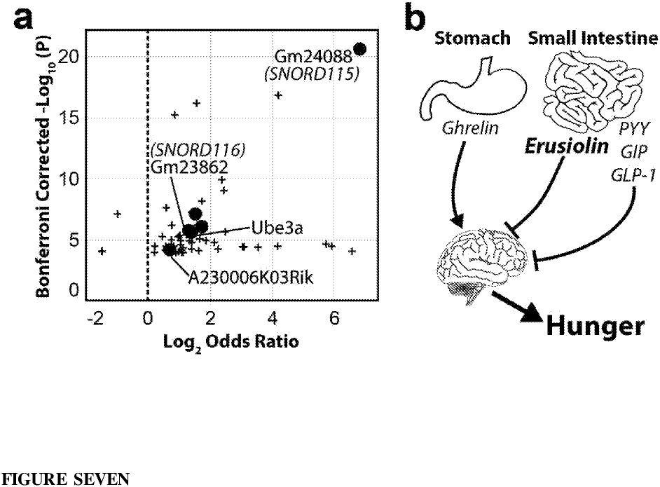

In this example, the Quantitative Endocrine Network Interaction Estimation method was used to identify potential target tissues for erusiolin. This method analyses the natural variation within the mouse erusiolin locus across the hybrid mouse diversity panel (HMDP), which consists of ~ 100 strains of mice, to search for correlated genes in global transcriptomic datasets from several tissues across the HMDP population. This highlighted the hypothalamus with many transcripts significantly linked to variation in the erusiolin locus (FIG. 7). Strikingly, a significant number of the transcripts were derived from the Prader-Willi Syndrome (PWS) locus on mouse chromosome 7 (FIG. 7a, black dots). PWS is a disease characterised by extreme hyperphagia (over-eating) and subsequent obesity. PWS patients typically have a large deletion (inside 15q11-q13) on their paternal chromosome 15, which when combined with the standard imprinting (silencing) of the maternal 15q11-q13 region, leads to loss of all transcription from genes inside this locus. Recent studies have identified the key gene in this region is SNORD116, whose loss of gene transcription alone is the causative event in PWS. This led to the hypothesis that the conserved peptide from erusiolin is secreted by the duodenum into blood plasma after feeding and acts on the hypothalamus to activate transcription of the PWS locus, leading to satiety (fullness) signals (FIG. 7b).

### Example Five: Structural Prediction for the Erusiolin Full Length Peptide

### 5.1 Erusiolin Peptide Synthesis and TFA Removal

The full-length human erusiolin peptide (APFLLEDPANQFLRLKR) was synthesised and purified as the TFA-salt by a commercial supplier (Genscript), which generated 4 mg of peptide at 99.9% purity. TFA removal and formation of the HCL-peptide salt was performed by freeze-drying the peptide solution (in LC-grade H₂O) for 16 h and resuspension in 500 uL of 10 mM HC1 in LC-grade H₂O. This solution was then freeze-dried for 16 h. A total of three rounds of 10 mM HC1 resuspension and freeze-drying for 16 h were performed. After the final drying step, the peptide was resuspended in LC-grade H₂O. The peptide solution was analysed for TFA contamination and determination of the peptide concentration using FT-IR spectroscopy. No TFA contamination was observed and the final peptide concentration was 1.04 mg/mL in LC-grade H₂O.

### 5.2 Circular Dichroism Spectropolarimetry (CD) Data Acquisition and Analysis

Far-UV spectra were measured at 20° C. on a CD spectrophotometer (Jasco J-720) using a 200 µm path length quartz cuvette. Data were collected every 1 nm with a 1 nm bandwidth in the 180-320 nm wavelength region using an integration time of 5 s per step. Protein samples were diluted to 0.15 mg/mL (75 µM) in 10 mM sodium phosphate buffer (pH 6.5) and the CD spectra measured between 260 and 190 nm. The resulting spectra represent the average of three accumulations and are buffer baseline corrected. Spectral smoothing (2^{nd} order) using 4 nearest neighbours was applied.

### 5.3 NMR Chemical Shift and NOE Data Acquisition and Analysis

NMR spectral acquisition and data analysis were as described previously (Mohanty B, et al. Proteins. 2019. 87(8):699-705. doi: 10.1002/prot.25687).

### 5.4 Results

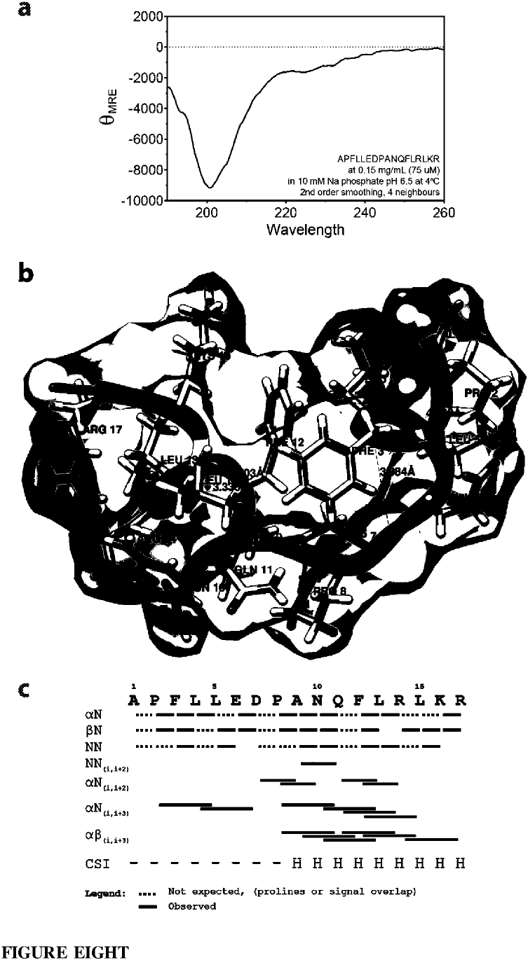

To examine the solution phase structure of the human erusiolin full length peptide (APFLLEDPANQFLRLKR), sold-phase peptide synthesis and subsequent HPLC purification was used to generate large amounts of high purity peptide for analysis. To examine if the peptide displayed any helical propensity in solution, we used circular dichroism spectropolarimetry (CD) (FIG. 8a). This analysis showed that at least some proportion of the erusiolin peptide molecules display a helical character as indicated by the minima at 208 and 222 nm. Structural prediction for the erusiolin conserved peptide using the PEP-FOLD3 software package demonstrated that the C-terminal region would likely be alpha-helical in solution (FIG. 8b). This agreed with additional secondary structural predictions from Phyre2.

To more closely examine the secondary structure characteristics of the erusiolin peptide we used NMR. The chemical shifts of alpha carbon protons along the peptide backbone are known to be predictive of secondary structure. Comparison of the observed these shifts for erusiolin with those of an unstructured ‘standard’ peptide enabled detection of a tendency for helical structure in the C-terminal half of the peptide (CSI, H = helix). In addition to the chemical shift analysis, we examined the nuclear Overhauser effects (NOEs) between adjacent protons of various peptide backbone atoms. NOEs refer to interactions of the protons through space and are typically limited to maximum distances of 5 angstroms. Long-range interactions (e.g. i, i+3), of NN, alphaN, or alpha-beta, indicate the presence of an alpha helix within the sequence ANQFLRLKR of erusiolin. Together these data show that erusiolin displays some structure in solution, without needing to be bound to its cognate receptor, similar to other secreted small protein hormones (e.g. insulin, GLP-1, GIP).

1. A peptide comprising or consisting of the amino acid sequence X1 X2 X3 X4 X5 X6 X7 X8 X9 X10 X11 X12 X13 X14 X15 X16 X17 wherein X1, X2, X16 and X17 are optionally present, and wherein:
X1 is selected from A, L, V, and I, or is absent;
X2 is selected from P and S, or is absent;
X3 is selected from F and V;
X4 is selected from L, M, V, I, A and F;
X5 is selected from L, M, S, K, L, A and T;
X6 is selected from E, Q, G and P;
X7 is selected from D, Q, K and E;
X8 is selected from P, S, E, A, Q and T;
X9 is selected from A, S, D, T and V;
X10 is selected from N and K;
X11 is selected from Q, R, G, K and E;
X12 is selected from F and I;
X13 is selected from L, I and M;
X14 is selected from R, H, G and Q;
X15 is selected from L, M, H, F and Q;
X16 is K or is absent; and
X17 is R or is absent.

2. The peptide according to claim 1, wherein:
X1 is A, or is absent;
X2 is P, or is absent;
X3 is F;
X6 is E;
X9 is A;
X12 is F;
X14 is R; and
X15 is L.

3. The peptide according to claim 2, wherein:
X4 is L;
X10 is N;
X11 is Q; and
X13 is L.

4. The peptide according to claim 1, wherein the peptide comprises or consists of an amino acid sequence as defined in any one of SEQ ID NO: 1, SEQ ID NO: 2, SEQ ID NO: 3 or SEQ ID NO: 4.

5. The peptide according to claim 1, wherein the peptide comprises one or more modifications to the chemical structure of one or more amino acids and/or peptide bonds.

6. The peptide according to claim 5, wherein the modification/s comprise:
N-terminal acetylation; and/or
N-methylation of peptide bonds and/or
replacement of an L-stereoisomer amino acid residue with the equivalent D-stereoisomer.

7. The peptide according to claim 5, wherein the modification/s protect a peptide bond from cleavage by a protease.

8. (canceled)

9. The peptide according to any claims 1, wherein:
the peptide is a synthetic peptide, and/or
the peptide is a hormone and/or is capable of hormone activity.

10. The peptide according to claim 9, wherein the hormone is capable of appetite regulation and/or produces satiety signals.

11. (canceled)

12. The peptide according to claim 1, wherein the peptide:
has at least 70%, at least 80%, at least 85%, at least 90%, at least 95% or 100% of the biological activity of a peptide having an amino acid sequence as defined in any one of SEQ ID NO: 1, SEQ ID NO: 2, SEQ ID NO: 3 or SEQ ID NO: 4, and /or-
has an increased biological activity of at least 10%, at least 20%, at least 30%, at least 40%, at least 50%, at least 60%, at least 70%, at least 80%, at least 90%, or at least 100% above the biological activity of a peptide having an amino acid sequence as defined in any one of SEQ ID NO: 1, SEQ ID NO: 2, SEQ ID NO: 3 or SEQ ID NO: 4.

13. (canceled)

14. An antibody that binds specifically to the peptide of claim 1.

15. The antibody according to claim 14, wherein the antibody is a polyclonal antibody.

16. The polyclonal antibody according to claim 15, wherein the polyclonal antibody is a rabbit polyclonal antibody.

17. The antibody according to claim 14, wherein the antibody is a monoclonal antibody.

18. The monoclonal antibody according to claim 17, wherein the monoclonal antibody is a rabbit monoclonal antibody.

19. A nucleic acid molecule encoding the peptide according to claim 1, wherein the nucleic acid molecule is operably linked to at least one heterologous regulatory element.

20. The nucleic acid molecule according to claim 19, wherein the nucleic acid molecule comprises at least one modification to enhance expression of the nucleic acid molecule in a heterologous host.

21. The nucleic acid molecule according to claim 20, wherein the at least one modification optimises the use of codons to enhance expression of the nucleic acid molecule in the heterologous host.

22. A vector comprising the nucleic acid molecule according to claim 19.

23. A host cell comprising the nucleic acid molecule according to claim 19 as a transgene and/or the vector according to claim 22.
